SEQUENCE LISTING (4 sequences)

SEQ ID NO: 1 - protein, 15 aa, synthetic construct
XYFAYHPNIPGLXYF

SEQ ID NO: 2 - protein, 15 aa, synthetic construct
YYFAYHPNIPGLYYF

SEQ ID NO: 3 - protein, 15 aa, synthetic construct
WYFAYHPNIPGLWYF

SEQ ID NO: 4 - protein, 15 aa, synthetic construct
FYFAYHPNIPGLFYF

(19) United States
(12) Patent Application Publication
(10) Pub. No.: US 2024/0408167 A1
(43) Pub. Date: Dec. 12, 2024

(54) COMPOSITION FOR TREATING OCULAR DISEASES CONTAINING PEPTIDE

(71) Applicant: Encuragen Inc., Anyang-si (KR)

(72) Inventors: Brian Byeoung KIM, Yongin-si (KR); Haksoon KIM, Suwon-si (KR); Inkyung LEE, Anyang-si (KR)

(21) Appl. No.: 18/413,660

(22) Filed: Jan. 16, 2024

(30) Foreign Application Priority Data
Jun. 8, 2023 (KR) 10-2023-0073646

(51) Int. Cl. A61K 38/10; A61K 9/00; A61K 47/60; A61P 27/02; A61P 27/04

(52) U.S. Cl. CPC A61K 38/10; A61K 9/0048; A61K 47/60; A61P 27/02; A61P 27/04

(57) ABSTRACT

A pharmaceutical composition for treating of ocular diseases, containing a peptide is described. The composition is excellent in improving tear volume, extending tear decomposition time, and exhibiting anti-inflammatory effect and anti-apoptotic effect, and at the same time, since it has excellent stability even in the form of eye drops, it can be used as a composition for treating various ocular diseases, including dry eye syndrome.

### CROSS-REFERENCE TO RELATED APPLICATIONS

This application claims the priority of Korean Patent Application No. 10-2023-0073646 filed on Jun. 8, 2023, in the Korean Intellectual Property Office, the disclosure of which is incorporated herein by reference in its entirety.

### INCORPORATION BY REFERENCE OF SEQUENCE LISTING

A Sequence Listing is provided herewith as a Sequence Listing XML. “9-PJK4966918. Sequence_231228” created on Oct. 23, 2023, and modified on Jan. 16, 2024, and having a size of 4.113 bytes. The contents of the Sequence Listing XML are incorporated herein by reference in their entirety.

### STATEMENT REGARDING PRIOR DISCLOSURES BY THE INVENTORS

The inventors of the present application are the authors of the article, “Enhanced Immunomodulation, Anti-Apoptosis, and Improved Tear Dynamics of (PEG)-BHD1028, a Novel Adiponectin Receptor Agonist Peptide, for Treating Dry Eye Disease” published on Dec. 26, 2022, one year or less before the effective filing date of the present application, which is not prior art under 35 U.S.C. 102 (b) (1) (A).

### BACKGROUND OF THE INVENTION

### Field of the Invention

The present invention relates to a composition for treating ocular diseases containing a peptide, and more specifically, to a pharmaceutical composition for treating ocular diseases containing a peptide effective for the treatment of dry eye syndrome.

### Description of the Related Art

Dry eye syndrome is a tear film disease that causes irritation in the eyes due to a lack of tears. Dry eye syndrome occurs due to decreased tear secretion, excessive evaporation of tears, or inflammation of the tear producing organ, or it occurs when accompanied by systemic diseases such as Sjögren's syndrome, Steven Johnson syndrome, and pemphigoid.

When dry eye syndrome occurs, the eyes become sore, and a foreign matter sensation like grains of sand in the eyes, and a prickly feeling occur. In addition, the eyes become more tired than usual, making it difficult to open the eyes properly. In particular, when facing a cold wind in the winter, tears will flow, and in severe cases, a headache is complained, and the eyes become bloodshot. Dry eye syndrome is a condition in which the body produces less tears, and it is difficult to completely cure. Among various treatment methods to ameliorate the symptoms and calm down the inflammatory responses, appropriate treatment tailored to a person's own eye conditions is provided. When dry eye is very severe, the cornea may dry out and the vision may be severely reduced.

Meanwhile, although it is difficult to cure dry eye syndrome completely, various treatment methods have been attempted. In a case of dry eye syndrome due to lack of an aqueous layer, artificial tears are dropped. In a case of increased tear evaporation due to lack of a fat layer, eyelid inflammation treatment is performed. In the case in which eye inflammation is the main cause, anti-inflammatory treatment is accompanied.

A drug to treat dry eye syndrome is cyclosporine (product name: Restasis), which was approved by the FDA in 2003, and it treats inflammation caused by dry eye syndrome by inhibiting the production of interleukin-2 in T cells. However, cyclosporine has problems that it may accompany side effects such as decreased renal functions, increased blood pressure, headache, hyperlipidemia, vomiting, hirsutism, gingival hyperplasia, and increased blood sugar, and that fundamental treatment of dry eye syndrome is difficult.

Recently, as a new drug for dry eye syndrome after cyclosporine, lifitegrast (product name: Xiidra) was released to the market, and it is known to prevent the activation of T cells by competitively binding to the adhesion molecules positioned on the surface of T cells, thereby inhibiting inflammation of dry eye syndrome. However, it has been reported that lifitegrast also cause adverse ophthalmic drug reactions such as ocular irritation, ocular pain, and reaction at the dripping site, and it is known that fundamental treatment dry eye syndrome is difficult.

Against this background, there is growing interest in therapeutics for dry eye syndrome that have excellent therapeutic efficacy and excellent safety.

### SUMMARY OF THE INVENTION

The present inventors prepared a composition for treating ocular diseases containing a peptide, and confirmed that a composition according to the present invention has a superior therapeutic efficacy in treating ocular diseases including dry eye syndrome.

Accordingly, an object of the present invention is to provide a composition for treating ocular diseases containing a peptide as an active ingredient.

Another object of the present invention is to provide a method for treating ocular diseases using a peptide.

Another object of the present invention is to provide an use of a peptide for treating ocular diseases.

The present invention relates to a composition for treating ocular diseases containing a peptide, and the composition according to the present invention has a superior excellent therapeutic efficacy and excellent safety in treating ocular diseases including dry eye. Hereinafter, the present invention will be described in more detail.

One aspect of the present invention is a pharmaceutical composition for treating, preventing, alleviating or suppressing ocular diseases, containing a peptide including SEQ ID NO: 1 (Xaa1-Tyr-Phe-Ala-Tyr-His-Pro-Asn-Ile-Pro-Gly-Leu-Xaa2-Tyr-Phe) as an active ingredient.

In the present invention, Xaa1 of SEQ ID NO: 1 may be any one selected from the group consisting of tyrosine, tryptophan, phenylalanine, and non-natural amino acids having the same properties as these.

In the present invention, Xaa2 of SEQ ID NO: 1 may be any one selected from the group consisting of tyrosine, tryptophan, phenylalanine, and non-natural amino acids having the same properties as these.

A peptide according to the present invention includes salt forms thereof. Examples of such salts include metal salts, ammonium salts, salts with organic bases, salts with inorganic acids, salts with organic acids, salts with basic or acidic amino acids, and the like. Preferred examples of metal salts include alkali metal salts such as sodium salt, potassium salt, and the like; alkaline earth metal salts such as calcium salts, magnesium salts, barium salts, and the like; and aluminum salts and others.

A peptide of the present invention may be produced according to a peptide synthesis method known in the art. As an example, it may be conversion to a peptide molecule of the present invention under physiological conditions. For example, a peptide of the present invention may be synthesized according to a solid-phase synthesis process and a liquid-phase synthesis process. That is, a peptide provided by the present invention may be produced by repeatedly condensing a partial peptide or amino acid that may constitute a peptide molecule, a peptide to be synthesized, and the remaining portion in a desired order. In the case in which a product with a desired sequence has a protecting group, the desired peptide may be produced by removing the protecting group.

In the present invention, a peptide may be in the form of a peptide with a carrier material linked thereto.

A carrier material may be selected from the group consisting of an immunoglobulin Fc region, a lipophilic compound, an amino acid, albumin, transferrin, and polyethylene glycol (PEG), but is not limited thereto.

PEG non-specifically binds to a specific region or various regions of a target peptide, and has an effect of increasing the molecular weight of the peptide, suppressing the loss by the kidneys, and preventing hydrolysis thereof without causing any particular side effects. Furthermore, when PEG is coupled with a foreign peptide, the recognition of an antigenic site present in the foreign peptide by immune cells may sometimes be inhibited. Specifically, phagocytosis and intracellular proteolysis of a peptide by antigen-presenting cells may be inhibited, thereby reducing the possibility that the peptide acts as an antigen.

The term “amino acid” in the present specification refers to a molecule having a specific structure in which an amine group (—NH₂) having basic properties and a carboxyl group (—COOH) having acidic properties coexist. When an amino group bound to one amino acid molecule reacts with a carboxyl group bound to another amino acid molecule, water and a new molecule consisting of and two amino acids are formed. The new molecule is referred to as a dipeptide, and the bond formed by the reaction is referred to as a peptide bond. The part with an amine group is referred to as N terminal, and the part with a carboxyl group is referred to as C terminal. In order for a protein to form, numerous peptide bonds must be formed. There exist 20 amino acids in nature, and 12 of them, which are glycine, alanine, arginine, asparagine, aspartate, cysteine, glutamate, glutamine, histidine, proline, serine, and tyrosine, are synthesized in our bodies from the foods we eat. The other eight, which are isoleucine, leucine, lysine, tryptophan, valine, methionine, phenylalanine and threonine, are not synthesized.

Non-natural amino acid refers to an amino acid that does not exist in nature, an amino acid synthesized or made by human. Specifically, iodinated tyrosine, methylated tyrosine, glycosylated serine, glycosylated threonine, azetidine-2-carboxylic acid, 3,4-dehydroproline, perthiaproline, canavanine, ethionine, norleucine, selenomethionine, animohexanoic acid, telluromethionine, homoallylglycine, and homopropargylglycine are included, and D-amino acids are included in non-natural amino acids.

Non-natural amino acid having the same characteristics refers to an non-natural amino acid having physically, chemically or functionally similar characteristics to natural amino acids, and it refers to an amino acid exhibiting a similar or same effect as a natural amino acid when it replaces a natural amino acid. According to one embodiment of the present invention, an amino acid having the same characteristics as tyrosine, tryptophan and phenylalanine may be an amino acid having aromatic characteristics. An aromatic amino acid refers to an amino acid having an aromatic ring (benzene ring and derivatives thereof) in a side chain of the amino acid. Therefore, it may be an non-natural amino acid having properties that are the same as or similar to an aromatic amino acid.

A peptide according to the present invention may be linked to a carrier material through a linker.

A linker may be a peptidic or non-peptidic polymer.

Non-peptidic polymers may be selected from the group consisting of polyethylene glycol, polypropylene glycol, copolymers of ethylene glycol and propylene glycol, polyoxyethylated polyol, polyvinyl alcohols, polysaccharides, dextran, polyvinyl ethyl ether, biodegradable polymers such as polylactic acid (PLA) and polylactic-glycolic acid (PLGA), lipid polymers, chitins, hyaluronic acid, and combinations thereof. Derivatives of non-peptide polymers already known in the art according to the present invention and derivatives that may be easily prepared at the skill level in the art may also be used as linkers.

A peptide according to the present invention may be modified by phosphorylation, sulfation, acrylation, glycosylation, methylation, farnesylation, acetylation, amidation or the like within a range in which the overall activity of the molecule is not changed.

In one embodiment of the present invention, a linking compound of X-L1 may be further linked to an N terminus of an amino acid sequence of SEQ ID NO: 1.

In one embodiment of the invention, X may be any one selected from the group consisting of 1 to 10 amino acids; polyethylene glycol in a linear or branched form having a weight of 1 to 200 kDa; a lipophilic compound; and a peptide transduction domain, for example, it may be polyethylene glycol, but is not limited thereto.

In one embodiment of the present invention, L1 may be a compound that plays the role of a single bond or a link connecting an N terminus of SEQ ID NO: 1 and the X above.

In one embodiment of the present invention, an L2-Z linking compound may be further included at a C terminus of the amino acid sequence of SEQ ID NO: 1.

In one embodiment of the invention, Z may be any one selected from the group consisting of 1 to 10 amino acids; polyethylene glycol in a linear or branched form having a weight of 1 to 200 kDa; a lipophilic compound; and a peptide transduction domain.

Polyethylene glycol (PEG) refers to a polymer of ethylene oxide. According to one embodiment of the present invention, polyethylene glycol may be a composition of methoxyl PEG maleimide (mPEG (MAL)), methoxyl PEG forked maleimide (mPEG2 (MAL)), methoxyl PEG ortho-pyridyldisulfide (mPEG-OPSS), PEG-vinylsulphone, or methoxyl PEG aldehyde (mPEG-ALD) and ortho-pyridyldisulfide-PEG-hydrazide (OPSS-PEG-hy-drazide). According to another embodiment of the present invention, the polyethylene glycol may be selected from the group consisting of 5k-mPEG (MAL), 20k-mPEG (MAL), 40k-mPEG2 (MAL), 5k-mPEG-OPSS, 10k-mPEG-OPSS, 20k-mPEG-OPSS, or mPEG30 kD-ALD and OPSS-PEG2k-hydrazide.

Lipophilic compounds may be compounds that exist in nature, specifically saturated or unsaturated fatty acids, fatty acid diketones, terpenes, prostaglandins, vitamins, carotenoids, steroids, or it may be a synthetic compound, specifically a carbon acid, an alcohol, an amine, and a sulfonic acid with one or more alkyl, aryl, alkenyl, or other unsaturated compounds.

A peptide transduction domain refers to a protein that can penetrate a cell membrane protein, and it may impart an ability to allow a complex formed by being combined with other compounds to enter into a cell membrane. According to one embodiment of the invention, a peptide transduction domain may be a protein having transmembrane properties that are known already.

In one embodiment of the present invention, L2 may be a compound that plays the role of a single bond or a link connecting a C terminus of SEQ ID NO: 1 and Z.

A compound that plays the role of a single bond or a link, that is, L1 or L2 may be a compound that is positioned between the X or the Z above and an N or C terminus of the amino acid sequence of the agonist peptide and that is capable of adding stability to the agonist peptide, and specifically, it may be at least two functional groups. L1 or L2 may be two or more functional groups such as an alkyl group, an aryl group, an arakyl group, or a peptide functional group.

In one embodiment of the present invention, SEQ ID NO: 1 may be any one selected from the group consisting of Tyr-Tyr-Phe-Ala-Tyr-His-Pro-Asn-Ile-Pro-Gly-Leu-Tyr-Tyr-Phe (SEQ ID NO: 2); Trp-Tyr-Phe-Ala-Tyr-His-Pro-Asn-Ile-Pro-Gly-Leu-Trp-Tyr-Phe (SEQ ID NO: 3); and Phe-Tyr-Phe-Ala-Tyr-His-Pro-Asn-Ile-Pro-Gly-Leu-Phe-Tyr-Phe (SEQ ID NO: 4).

The term “ocular disease” in the present specification refers to disorders and conditions affecting the eyes. In one embodiment of the present invention, an ocular disease may be one or more diseases selected from the group consisting of keratoconjunctivitis sicca (KCS), atopic keratoconjunctivitis (AKC), vernal keratoconjunctivitis (VKC), glaucoma, keratitis, corneal epithelium erosion, uveitis, intraocular inflammation, dry eye syndrome, dry-eye syndrome ocular infections, ocular infections, ocular allergy, corneal or conjunctival lesions, diabetic macular edema, and age-related macular degeneration. For example, an ocular disease may be dry eye syndrome or dry-eye syndrome ocular infections, but is not limited thereto.

The content of an active ingredient in the composition according to the present invention may be appropriately adjusted depending on the type and purpose of use, patient condition, the type and severity of symptoms, or the like.

The daily dosage of a composition according to the present invention may be appropriately adjusted depending on the type and purpose of use, patient condition, the type and severity of symptoms, or the like, and may be 1 to 1000 μg/ml based on the content of the active ingredient, for example, it may be 0.001 to 10000 mg/kg, but is not limited thereto.

The composition according to the present invention may be administered to mammals, including humans, by various routes. The administration method may be any commonly used method, for example, it may be administered orally, percutaneously, intravenously, intramuscularly, subcutaneously, ocularly, or through a route like that. For example, it may be administered intraocularly.

In the present invention, “prevention” refers to any action that suppresses or delays onset of an ocular disease by administering a composition according to the present invention.

In the present invention, “treatment” refers to any action in which symptoms of an ocular disease are ameliorated or beneficially changed by administering a composition according to the present invention.

A pharmaceutical composition of the present invention may be used as a single agent, and may be prepared and used as a composite agent by further including a pharmaceutical composition known to have an authorized ocular disease prevention or treatment effect. It may be formulated into a pharmaceutical unit dosage form by adding a pharmaceutically acceptable carrier, excipient, or diluent.

In the present invention, “pharmaceutically acceptable” means that it does not significantly stimulate a living organism and it does not inhibit the biological activity and properties of the administered active substance.

In the present invention, a pharmaceutical composition including a pharmaceutically acceptable carrier may have any one formulation selected from the group consisting of eye drops, artificial tears, gels, ointments, tablets, pills, powders, granules, capsules, suspensions, oral solutions, emulsions, syrups, sterilized aqueous solutions, non-aqueous solutions, suspensions, emulsions, freeze-dried preparations, and suppositories.

Solid preparations for oral administration include tablets, pills, powders, granules, capsules, etc., and these solid preparations may be prepared by mixing at least one excipient, such as starch, calcium carbonate, sucrose or lactose, and gelatin, with one or more compounds. In addition, besides a simple excipient, lubricants such as magnesium stearate and talc may also be used. Liquid preparations for oral administration correspond to suspensions, oral solutions, emulsions, and syrups, and in addition to commonly used simple diluents such as water and liquid paraffin, various excipients such as wetting agents, sweeteners, fragrances, and preservatives may be included.

Preparations for parenteral administration may include sterilized aqueous solutions, non-aqueous solutions, suspensions, emulsions, freeze-dried preparations, and suppositories. As non-aqueous solvents and suspensions, propylene glycol, polyethylene glycol, vegetable oils such as olive oil, and injectable esters such as ethyl oleate may be used. As a base material of a suppository witepsol, macrogol, tween 61, cacao oil, laurel oil, glycerogelatin or the like may be used, but it is not limited thereto. Specifically, eye drops, eye fluids or the like for intraocular administration may be included.

A pharmaceutical composition according to the present invention may include ethylene-diamine-tetraacetic acid (EDTA). Ethylenediaminetetraacetic acid may be included in a pharmaceutical composition as an antioxidant, and it is capable of preventing decomposition of protein drugs, which are vulnerable to oxidation. In a specific example of the present invention, it was confirmed that ethylene-diamine-tetraacetic acid may increase the stability of a peptide included in a pharmaceutical composition without causing decomposition of the sample, unlike sodium bisulfate, which weakens the stability of the composition (see Example 9).

In one embodiment of the invention, the pharmaceutical composition may include 0.01% to 1%, 0.1% to 0.9%, 0.15% to 0.85%, 0.2% to 0.8%, 0.25% to 0.75%, 0.3% to 0.7%, 0.35% to 0.65%, 0.4% to 0.6%, 0.45% to 0.55% (w/v), for example, 0.5% (w/v) of ethylene-diamine-tetraacetic acid, but it is not limited thereto.

A pharmaceutical composition according to the present invention may have a viscosity intermediate between that of normal people and dry eye syndrome. Specifically, a pharmaceutical composition according to the present invention may have a viscosity of 1 to 20 cP, 2 to 18 cP, 3 to 17 cP, 4 to 16 cP, 5 to 15 cP, 6 to 14 cP, 7 to 13 cP, 8 to 12 cP, for example, 10 cP. Ingredients for adjusting the viscosity of the pharmaceutical composition may include hyaluronic acid or hyaluronic acid salt. In a specific example of the present invention, it was confirmed that hyaluronic acid may achieve the viscosity without weakening the stability of the composition (see Example 9). In addition, a pharmaceutical composition according to one embodiment may include hyaluronic acid or hyaluronic acid salt at a concentration of 0.01% to 1%, 0.05% to 0.95%, 0.05% to 0.90%, 0.05% to 0.8%, 0.05% to 0.7%, 0.05% to 0.6%, 0.05% to 0.5%, 0.05 to 0.4%, 0.05% to 0.3%, 0.05% to 0.2%, 0.06% to 0.15%, 0.08% to 0.15%, 0.08% to 0.12%, or 0.1% (w/v), but it is not limited thereto.

The pharmaceutical composition according to the present invention may have a pH of 5.0 to 9.0, 5.2 to 8.8, 5.4 to 8.6, 5.6 to 8.6, 5.8 to 8.4, 6.0 to 8.2, 6.0 to 8.0, 6.2 to 7.8 or 6.5 to 7.6. Ingredients for pH adjustment may include water, hydrochloric acid (HCl), sodium hydroxide (NaOH), sodium phosphate buffer, and/or sodium azide.

In one embodiment of the present invention, a pharmaceutical composition may have an osmolality of 280 to 340 mOsm/kg, 290 to 330 mOsm/kg, 290 to 320 mOsm/kg, 280 to 310 mOsm/kg or 290 to 310 mOsm/kg. As a sample to adjust osmotic pressure, trehalose and/or mannitol may be used. In a specific example of the present invention, it was confirmed that trehalose or mannitol single formulations and trehalose/mannitol composite formulations have better stability compared to formulations prepared by adding other samples such as salts and PEG.

In one embodiment of the invention, the pharmaceutical composition may include trehalose at a concentration of 1.0% to 20.0%, 2.0% to 18.0%, 3.0% to 17.0%, 4.0% to 16.0%, 5.0% to 15.0%, 6.0% to 14.0%, 7.0% to 13.0%, 8.0% to 12.0%, 7.0% to 11.0%, 8.0% to 10.0%, 8.2% to 9.8%, 8.4% to 9.6%, 8.6% to 9.4%, 8.8% to 9.0% or 9.0% (w/v), but it is not limited thereto.

In one embodiment of the invention, the pharmaceutical composition may include a peptide at a concentration of 0.1% to 5%, 0.1% to 4.5%, 0.1% to 4.0%, 0.1% to 3.5%, 0.1% to 3 17.0%, 0.1% to 2.5%, 0.1% to 2.0%, 0.1% to 1.0%, 0.1% to 0.9%, 0.1% to 0.8%, 0.1% to 0.7%, 0.1% to 0.6%, 0.2% to 0.8%, 0.3% to 0.7%, 0.4% to 0.6% or 0.5% (w/v). In a pharmaceutical composition according to the present invention, it was confirmed that when a peptide according to SEQ ID NO: 1 is included at a concentration of 0.5% to 1.5% or 0.5%, the osmotic pressure may be maintained at about 300 mOsm/kg, which is an appropriate osmolality of an eye drop preparation, and a viscosity of about 10 cP, which is intermediate between normal people and dry eye syndrome, may be achieved.

Another aspect of the present invention provides a method for preventing or treating an ocular disease, comprising a step of administering a pharmaceutical composition to a subject suspected of having an ocular disease.

In the present invention, “subject” refers to all animals including humans, monkeys, cows, horses, sheep, pigs, chickens, turkeys, quails, cats, dogs, mice, rats, rabbits, or guinea pigs in which an ocular disease has developed or may develop, and the ocular disease may be effectively prevented or treated by administering a pharmaceutical composition of the present invention to the subject. In addition, a pharmaceutical composition of the present invention may exhibit a synergistic effect when administered in combination with an existing therapeutic agent.

In the present invention, “administration” refers to providing a predetermined substance to a patient by any appropriate method, and the administration route of a composition of the present invention may be any general route as long as it may reach a target tissue. It may be administered intraocularly, intraperitoneally, intravenously, intramuscularly, subcutaneously, intradermally, orally, locally, intranasally, intrapulmonaryly, or rectally, but it is not limited thereto. In addition, a pharmaceutical composition of the present invention may be administered by any device capable of transporting an active substance to target cells. In addition, the administration method and formulation may be intravenous injection, subcutaneous injection, intradermal injection, intramuscular injection, drip injection, etc. An injection may be prepared by using aqueous solvents such as physiological saline solution and Ringer's solution, non-aqueous solvents such as vegetable oil, higher fatty acid esters (e.g., ethyl oleate, etc.), and alcohols (e.g., ethanol, benzyl alcohol, propylene glycol, glycerin, etc.), and it may include pharmaceutical carriers such as stabilizers to prevent deterioration (e.g., ascorbic acid, sodium bisulfite, sodium pyrosulphite, BHA, tocopherol, EDTA, etc.), emulsifiers, buffers for pH adjustment, and preservatives to prevent microbial growth (e.g., phenylmercuric nitrate, thimerosal, benzalkonium chloride, phenol, cresol, benzyl alcohol, etc.).

Another use of the present invention is the use of a pharmaceutical composition for treating an ocular disease.

The present invention relates to a pharmaceutical composition for treating an ocular disease, including an agonist peptide for an adiponectin receptor. An composition according to the present invention is excellent in improving tear volume, extending tear decomposition time, and exhibiting anti-inflammatory effect and anti-apoptotic effect, and at the same time, since it has excellent stability even in the form of eye drops, it can be used as a composition for treating various ocular diseases, including dry eye syndrome.

### BRIEF DESCRIPTION OF THE DRAWINGS

FIG. 1 shows a graph of confirming the results of the tear production of the control group (EDE), vehicle group (PBS), and (PEG)-BHD1028 administration groups (0.001%, 0.01%, and 0.1%) in the mouse model through a phenol red thread test on Day 5 and Day 10.

FIG. 2 shows a graph of the Schirmer tear test (STT) results of the vehicle group (PBS), (PEG)-BHD1028 administration groups (0.1%, 0.2%, and 0.4%), and cyclosporine administration group (0.05%) in the rabbit model on Day 5 and Day 10.

FIG. 3 shows a graph of the Schirmer tear test (STT) results of the vehicle group (PBS), (PEG)-BHD1028 administration groups (0.1%, 0.2%, and 0.4%), and cyclosporine (0.05%) administration group in the rabbit model at the baseline and Day 0, Day 5, and Day 10.

FIG. 4 shows a graph of the results of measuring the tear film break-up time (TBUT) of the control group (EDE), vehicle group (PBS), and (PEG)-BHD1028 administration groups (0.001%, 0.01%, and 0.1%) in the mouse model on Day 5 and Day 10.

FIG. 5 shows a graph of the results of measuring the TBUT of the vehicle group (PBS), (PEG)-BHD1028 administration groups (0.1%, 0.2%, and 0.4%), and cyclosporine administration group (0.05%) in the rabbit model on Day 5 and Day 10.

FIG. 6 shows a graph of the results of measuring the TBUT of the vehicle group (PBS), (PEG)-BHD1028 administration groups (0.1%, 0.2%, and 0.4%), and cyclosporine (0.05%) administration group in the rabbit model at the baseline and Day 0, Day 5, and Day 10.

FIGS. 7A-7B show a graph and photographs of the results of measuring the clinical severity using a corneal fluorescent staining (CFS) method of the control group (EDE), vehicle group (PBS), and (PEG)-BHD1028 administration groups (0.001%, 0.01%, and 0.1%) in the mouse model on Day 5 and Day 10.

FIG. 8 shows a graph of the results of measuring the CSS of the vehicle group (PBS), (PEG)-BHD1028 administration groups (0.1%, 0.2%, and 0.4%), and cyclosporine (0.05%) administration group in the rabbit model on Day 5 and Day 10.

FIG. 9 shows a graph of the results of measuring the CSS of the vehicle group (PBS), (PEG)-BHD1028 administration groups (0.1%, 0.2%, and 0.4%), and cyclosporine (0.05%) administration group in the rabbit model at the baseline and Day 0, Day 5, and Day 10.

FIGS. 10A-10C show a diagram of and photographs of the results of measuring clinical severity using a corneal fluorescent staining (CFS) method of the vehicle group (PBS), (PEG)-BHD1028 administration groups (0.1%, 0.2%, and 0.4%), and cyclosporine (0.05%) administration group in the rabbit model at the baseline and Day 0, Day 5, and Day 10.

FIG. 11 shows a diagram of the results of measuring the CD4+IFN-γ+T cell density in the cornea, conjunctiva, and lacrimal gland in order to measure the anti-inflammatory effect of the control group (EDE), vehicle group (PBS), and (PEG)-BHD1028 administration groups (0.001%, 0.01%, and 0.1%) in the mouse model.

FIGS. 12A-12C show a graph of the results of measuring the CD4+IFN-γ +T cell density in the cornea, conjunctiva, and lacrimal gland in order to measure the anti-inflammatory effect of the control group (EDE), vehicle group (PBS), and (PEG)-BHD1028 administration groups (0.001%, 0.01%, and 0.1%) in the mouse model.

FIG. 13 shows a diagram of the results of measuring the CD11b+T cell counts in the cornea, conjunctiva, and lacrimal gland in order to measure the anti-inflammatory effect of the control group (EDE), vehicle group (PBS), and (PEG)-BHD1028 administration groups (0.001%, 0.01%, and 0.1%) in the mouse model.

FIGS. 14A-14C show a graph of the results of measuring the CD11b+T cell counts in the cornea, conjunctiva, and lacrimal gland in order to measure the anti-inflammatory effect of the control group (EDE), vehicle group (PBS), and (PEG)-BHD1028 administration groups (0.001%, 0.01%, and 0.1%) in the mouse model.

FIGS. 15A-15B show the results of confirming the immune cell counts in the corneal stroma of the vehicle group (PBS), (PEG)-BHD1028 administration groups (0.1%, 0.2%, and 0.4%), and cyclosporine administration group (0.05%) in the DED rabbit model through H&E.

FIGS. 16A-16B show the results of confirming the immune cell counts in the conjunctiva of the vehicle group (PBS), (PEG)-BHD1028 administration groups (0.1%, 0.2%, and 0.4%), and cyclosporine administration group (0.05%) in the DED rabbit model through H&E.

FIGS. 17A-17B show the results of confirming the immune cell counts in the lacrimal gland of the vehicle group (PBS), (PEG)-BHD1028 administration groups (0.1%, 0.2%, and 0.4%), and cyclosporine administration group (0.05%) in the DED rabbit model through H&E.

FIGS. 18A-18B show a diagram of the results of confirming the corneal thickness of the vehicle group (PBS), (PEG)-BHD1028 administration groups (0.1%, 0.2%, and 0.4%), and cyclosporine administration group (0.05%) in the DED rabbit model.

FIGS. 19A-19C show a diagram of the results of performing a TUNEL analysis in order to measure the antiapoptotic effect of the control group (EDE), vehicle group (PBS), and (PEG)-BHD1028 administration groups (0.001%, 0.01%, and 0.1%) in the mouse model.

FIGS. 20A-20B show a diagram of the results of detecting apoptotic cells in the cornea of the vehicle group (PBS), (PEG)-BHD1028 administration groups (0.1%, 0.2%, and 0.4%), and cyclosporine administration group (0.05%) in the DED rabbit model [(a) Control (normal); (b) vehicle; (c) 0.05% Cyclosporine; (d) 0.1% (PEG)-BHD1028; (e) 0.2% (PEG)-BHD1028; and (f) 0.4% (PEG)-BHD1028].

FIGS. 21A-21B show a diagram of the results of detecting apoptotic cells in the conjunctiva of the vehicle group (PBS), (PEG)-BHD1028 administration groups (0.1%, 0.2%, and 0.4%), and cyclosporine administration group (0.05%) in the DED rabbit model [(a) Control (normal); (b) vehicle; (c) 0.05% Cyclosporine; (d) 0.1% (PEG)-BHD1028; (e) 0.2% (PEG)-BHD1028; and (f) 0.4% (PEG)-BHD1028].

FIGS. 22A-22B show a graph of the results of comparatively analyzing all test results (STT-2, TBUT, CFS, immune cell counts) of 0.4% (PEG)-BHD1028 and 0.05% cyclosporine in the rabbit EDE model on Day 10 using Student's t-test.

FIGS. 23A-23B show a diagram of the results of evaluating the stability when sodium bisulfate was set as an antioxidant for eye drops using (PEG)-BHD1028, stored at 40° C. for 7 days, and then analyzed.

FIGS. 24A-24B show a diagram of the results of evaluating the stability when sodium EDTA (0.5%) was set as an antioxidant for eye drops using (PEG)-BHD1028, stored at 40° C. for 25 days, and then analyzed.

FIGS. 25A-25B show a diagram of the results of evaluating the stability of eye drops including no sodium EDTA (0.5%) as an antioxidant for the eye drops using (PEG)-BHD1028 after storing it at 40° C. for 25 days.

FIGS. 26A-26B show a diagram of the results of evaluating the stability on Day 0 by including 0.1% hyaluronic acid in the eye drops using (PEG)-BHD1028.

FIGS. 27A-27B show a diagram of the results of evaluating the stability on Day 7 by including 0.1% hyaluronic acid in the eye drops using (PEG)-BHD1028.

FIGS. 28A-28B show a diagram of the results of evaluating the stability on Day 30 by including 0.1% hyaluronic acid in the eye drops using (PEG)-BHD1028.

FIGS. 29A-29B show a diagram of the results of evaluating the stability on Day 37 by including 0.1% hyaluronic acid in the eye drops using (PEG)-BHD1028.

FIGS. 30A-30D show a diagram of the results of testing the stability of eye drops using (PEG)-BHD1028 by using water (top) and PBS (bottom) as a diluent.

FIGS. 31A-31D show a diagram of the results of testing the stability of eye drops using (PEG)-BHD1028 by using KCL (top) and NaCl (bottom) as an osmotic pressure retention agent.

FIGS. 32A-32F show a diagram of the results of testing the stability of eye drops using (PEG)-BHD1028 by using PEG400 (top), PEG5000 (middle), and PEG3350 (bottom) as an osmotic pressure retention agent.

FIGS. 33A-33B show a diagram of the results of testing the stability of eye drops using (PEG)-BHD1028 by using trehalose as an osmotic pressure retention agent.

FIGS. 34A-34B show a diagram of the results of testing the stability of eye drops using (PEG)-BHD1028 by using mannitol as an osmotic pressure retention agent (stored at 40° C. for 6 days).

FIGS. 35A-35B show a diagram of the results of testing the stability of eye drops using (PEG)-BHD1028 by compositely using trehalose and mannitol as an osmotic pressure retention agent.

FIG. 36 shows a diagram of the results of evaluating the degree of the corneal damage amelioration of the vehicle group (PBS), eye drop vehicle group (Formulated Vehicle), and (PEG)-BHD1028 eye drop group (0.5% (PEG)-BHD1028 administration group).

FIG. 37 shows a diagram of the results of evaluating the degree of the corneal thickness of the vehicle group (PBS), eye drop vehicle group (Formulated Vehicle), and (PEG)-BHD1028 eye drop group (0.5% (PEG)-BHD1028 administration group).

FIG. 38 shows a diagram of the results of confirming the immune cell counts in the corneal stroma of the vehicle group (PBS), eye drop vehicle group (Formulated Vehicle), and (PEG)-BHD1028 eye drop group (0.5% (PEG)-BHD1028 administration group).

FIG. 39 shows a diagram of the results of confirming the immune cell counts in the conjunctival stromal of the vehicle group (PBS), eye drop vehicle group (Formulated Vehicle), and (PEG)-BHD1028 eye drop group (0.5% (PEG)-BHD1028 administration group).

FIG. 40 shows a diagram of the results of confirming the immune cell counts in the lacrimal gland of the vehicle group (PBS), eye drop vehicle group (Formulated Vehicle), and (PEG)-BHD1028 eye drop group (0.5% (PEG)-BHD1028 administration group).

FIG. 41 shows a diagram of the results of measuring the antiapoptotic effect in the corneal epithelial of the vehicle group (PBS), eye drop vehicle group (Formulated Vehicle), and (PEG)-BHD1028 eye drop group (0.5% (PEG)-BHD1028 administration group).

FIG. 42 shows a diagram of the results of measuring the antiapoptotic effect in the conjunctiva of the vehicle group (PBS), eye drop vehicle group (Formulated Vehicle), and (PEG)-BHD1028 eye drop group (0.5% (PEG)-BHD1028 administration group).

FIG. 43 shows a diagram of the results of measuring the antiapoptotic effect in the conjunctival goblet cell of the vehicle group (PBS), eye drop vehicle group (Formulated Vehicle), and (PEG)-BHD1028 eye drop group (0.5% (PEG)-BHD1028 administration group).

### DETAILED DESCRIPTION OF THE EMBODIMENTS

Hereinafter, the present invention will be described in more detail through the examples described below. However, these examples are only for illustrating the present invention, and the scope of the present invention is not limited by these examples.

### Example 1: Materials and Methods

### 1-1. Synthesis of (PEG)-BHD1028

The amino acid sequence of (PEG)-BHD1028 is NH₂-Tyr-Tyr-Phe-Ala-Tyr-His-Pro-Asn-Ile-Pro-Gly-Leu-Tyr-Tyr-Phe-COOH. The preparation process of (PEG)-BHD1028 consists of two steps: (1) peptide synthesis and (2) PEGylation and salt exchange

The peptide was synthesized by the Fmoc (9-fluorenylmethoxycarbonyl) solid-phase synthesis process, beginning with swelling and coupling of an anchor resin (H-Phe-2Cl-Trt-Resin). The C-terminus of the first amino acid was mixed with HBTU/N-Methylmorpholine/DMF and connected to the anchor resin. Then, Fmoc was deprotected using piperidine/dimethylformamide (DMF). Amino acids were linked to the growing chain after activating the carboxylic acid terminus. This process was repeated until the last N-terminal tyrosine amino acid was attached. After the synthesis ended, the allyloxycarbonyl (alloc) amino acid protecting group of the resin was removed from the peptide by treating with trifluoroacetic acid (TFA). The resulting peptide was precipitated in ether to obtain a crude linear peptide as an intermediate. The intermediate peptide was purified and concentrated by reverse phase HPLC. The crude PEGylated peptides PEGylated with 5 kDa methoxy-polyethylene glycol (mPEG) underwent primary purification and reflux purification by RP-HPLC, followed by acetate exchange using RP-HPLC. Afterwards, the salt exchanged peptide was freeze-dried. In order to confirm the chemical properties of (PEG)-BHD1028, the structure and full mass of the API batch were confirmed using FT-IR spectroscopy (FT-IR 4600, JASCO, Japan) and a MALDI-TOF MS analysis (ABSCIEX TOF/TOF™ 5800, USA).

### 1-2. Animal Experiment

The experimental protocol and animal care complied with the Guide for the Laboratory Animal Care and Use and were approved by the Institutional Animal Care and Use Committee of Chonnam National University. The date and code of the animal study approval were Feb. 16, 2021 and CNUHIACUC-21009 for the mice and Sep. 28, 2021 and CNU IACUC-YB-2021-123 for the rabbits. In the present study, 8-week-old female C57BL/6 mice and New Zealand white rabbits (Damool Science, Korea) with a weight of 2 to 3 kg were used.

### 1-3. Study Design and DED Modeling

To confirm the pharmacological effect and appropriate dosage, a pilot study was first conducted with the mice. In addition, a rabbit DED (Dry Eye Disease) model with a large exposed facial area was evaluated in comparison with a mouse model. In the mouse experimental dry eye (EDE) model, scopolamine hydrobromide (Sigma-Aldrich. St. Louis, MO, USA) was subcutaneously injected three times a day (9 a.m., 1:30 p.m., 6 p.m.) in a room exposed to ventilation with an ambient humidity of less than 40% at 25±2° C. to induce DED.

The mice were randomly divided into five groups: (1) EDE (experimental dry eye) control mice without eye drops administration, (2) EDE mice treated with a vehicle (phosphate-buffered saline), (3) EDE mice treated with 0.001% (PEG)-BHD1028 (4) EDE mice treated with 0.01% (PEG)-BHD1028, and (5) EDE mice treated with 0.1% (PEG)-BHD1028. All the treatment groups received 2 μL of eye drops three times a day.

The rabbit DED model was induced by locally administrating 0.1% benzalkoniumchloride (BAC) drops (Sigma, St. Louis, MO, USA) twice a day (8 a.m. and 4 p.m.). Before the BAC treatment, the control group (baseline, naive) underwent standard dry eye clinical tests such as the Schirmer tear test (STT), TBUT, and corneal staining score (CSS) test. After the BAC treatment, the induction of dry eye syndrome was confirmed, and the test substance was administered for 10 days while maintaining the induction of dry eye syndrome. The rabbits were randomly divided into five groups: (1) EDE rabbits treated with a vehicle (phosphate-buffered saline), (2) EDE rabbits treated with 0.1% (PEG)-HD1028, (3) EDE rabbits treated with 0.2% (PEG)-HD1028, (4) EDE rabbits treated with 0.4% (PEG)-HD1028, and (5) EDE rabbits treated with 0.05% cyclosporine (Kukje Pharm Co., Ltd., Seongnam, Korea). The test substance was locally applied bis in die (BID) to both eyes.

### 1-4. Measurement of Tear Volume

The tear secretion was measured for 20 seconds by contacting a cotton thread containing phenol red (Zone-Quick™; Oasis Medical, Inc., Glendora, CA, USA) to the conjunctival sac on the lateral canthus of the mouse eye. After measuring the length using a Nikon SMZ1500 microscope, the length substituted to the prescribed formula, and the value was converted to volume. The tear production of the rabbits was measured by a modified Schirmer tear test using Whatman 41 filter paper strips (Tianjin Jingming New Technology Development Co., Ltd., Tianjin, China) on Day 0, Day 5, and Day 10. Eye examination of the rabbits was performed under anesthesia. The anesthesia was performed under sedation by performing intramuscular injection of xylazine (3 mg/kg, Rompun®; Bayer Korea, Korea) and intramuscular injection of 10 mg/kgtiletamine/zolazepam (Zoletil®, Virbac Laboratories, Carros, France). After adding 1 drop of proparacaine HCl 0.5% (Alcaine®, Alcon Korea Ltd., Seoul, Korea), excess tears and eye drops were removed with Weck-cel® cellulose eye drops. The wet length (mm) of the paper strip was read after 5 minutes.

### 1-5. Measurement of Tear Break-Up Time (TBUT)

TBUT is the time elapsed between the last blink and the first appearance of fluorescein decomposition, and it is an indicator of tear film stability. TBUT was evaluated by administering 1 μL of 1% fluorescein sodium dye to the lower conjunctival sac of the mice and visualizing the tear film using cobalt blue light of a slit-lamp biomicroscope. The measurement was repeated three times to obtain an average value.

### 1-6. Corneal Fluorescence Staining Evaluation

The subjects were evaluated by corneal staining, and the abnormal findings of the eyelids, cornea, and conjunctiva were recorded. After dropping 1 μL of the 1% fluorescent dye solution and washing with saline solution, and the cornea was observed under a slit-lamp microscope and scored to evaluate the degree of epithelial damage (degree of fluorescent staining). Corneal staining was graded using the NEI staining grid on a scale of 0 to 3 (0=normal, 1=mild, 2=moderate, 3=severe) in five corneal regions (nasal, central, temporal, inferior, and superior) with a maximum total score of 15 points.

### 1-7. Evaluation of Ocular Surface Inflammation in Mice

The cornea, conjunctiva, and each tissue required for analysis were excised with scissors, incised, and shaken with 0.5 mg/mL collagenase type D at 37° C. for 1 hour. After pulverizing the tissue, the resulting mixture was passed through a cell strainer, centrifuged, and resuspended in phosphate-buffered saline containing 1% bovine serum albumin.

### 1-8. Histopathological Evaluation of Ocular Surface Inflammatory Cell Infiltration in Rabbits

The extracted eyes were washed with phosphate-buffered saline (PBS), fixed in the modified Davidson's solution, and embedded in paraffin (Histocore Arcadia, Leica, Wetzlar, Germany). Afterwards, paraffin blocks were sliced into sections of a ˜6 μm thickness, mounted on microscope slides (HistoCore AUTOCUT, Leica, Germany), air dried, and stained with hematoxylin-eosin. The stained tissue slides were imaged, and data were obtained using an optical microscope/digital slide scanner (Axio Scan.Z1, Zeiss, Oberkochen, Germany). Image measurement was performed using ZEN (Zeiss, Germany) and ImageJ (NIH, Bethesda, MA, USA) software programs.

### 1-9. Apoptosis Evaluation

The eyes and appendages from each group were excised, fixed in the modified Davidson's solution, and embedded in paraffin, and the tissue sections were TUNEL-stained. In each section, the degree of apoptosis in the cornea and conjunctiva was measured using ZEN (Zeiss, Oberkochen, Germany) and Image J v1.51j8 (NIH, Bethesda, MD, USA) software programs.

### 1-10. Statistical Analysis

The variability of the results was expressed as mean±standard error of the mean (SEM), a p-value <0.05 was considered significant. A statistical analysis was performed using Student's t-test or one-way analysis of variance followed by Dunnett's multiple comparisons using GraphPad Prism 5.0 (GraphPad Software, San Diego, CA, USA).

### Example 2: Day 0 Ophthalmic Examination Baseline for Experimental Dry Eye (EDE)

Before starting the treatment (Day 0), ophthalmological examinations, including tear volume, tear film breakup time, and corneal fluorescein staining, were performed in the healthy (baseline) and EDE animals for grouping, and the results showed no significant discrepancies within each group.

### Example 3: Evaluation of Tear Production in Each EDE Animal Model

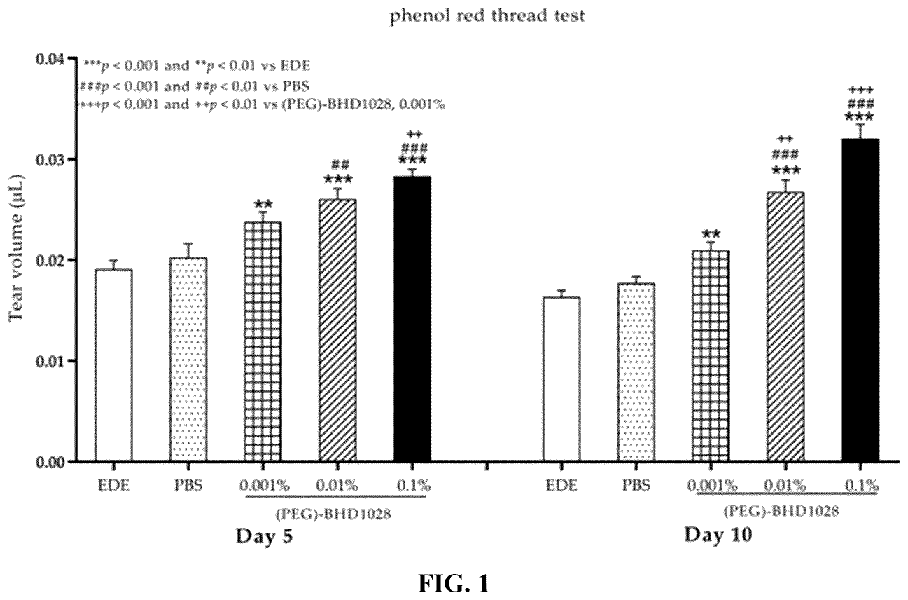

The tear volume of the mice was measured using the Phenol Red Thread (PRT) test on Day 5 and Day 10 after the treatment. On Day 5 and Day 10, the tear volume was significantly increased in all the three (PEG)-BHD1028 treatment groups compared to the EDE animals. The tear volume in the 0.01% and 0.1% (PEG)-BHD1028 groups was significantly more than the vehicle group from Day 5 (0.01% group: p<0.01 and 0.1% group: p<0.001 vs. vehicle), and it increased dose-dependently on Day 10 (0.01% group: p<0.01 and 0.1% group: p<0.001 vs. 0.001% group) (see FIG. 1 and Table 1).

TABLE 1

| | Period after | | | | | |
|---|---|---|---|---|---|---|
| | inducing | | | | | |
| Mouse | dry eye | | Vehicle | (PEG)-BHD1028 | | |
| model | syndrome | EDE | (PBS) | 0.001% | 0.01% | 0.1% |
| Tear | Day 5 | 0.019 ± 0.002 | 0.020 ± 0.004 | 0.024 ± 0.003 | 0.026 ± 0.003 | 0.028 ± 0.002 |
| volume | Day 10 | 0.016 ± 0.002 | 0.018 ± 0.002 | 0.021 ± 0.002 | 0.027 ± 0.003 | 0.032 ± 0.004 |
| (μL) | | | | | | |
| TBUT | Day 5 | 1.304 ± 0.086 | 1.324 ± 0.114 | 1.333 ± 0.081 | 1.406 ± 0.082 | 1.408 ± 0.095 |
| (sec) | Day 10 | 1.193 ± 0.092 | 1.229 ± 0.060 | 1.359 ± 0.140 | 1.530 ± 0.137 | 1.483 ± 0.113 |
| CFS | Day 5 | 10.875 ± 2.357 | 10.250 ± 2.435 | 9.875 ± 1.808 | 8.625 ± 1.598 | 8.125 ± 1.553 |
| (score) | Day 10 | 12.375 ± 1.923 | 12.625 ± 1.302 | 11.000 ± 1.927 | 7.625 ± 1.598 | 8.375 ± 1.302 |

In the rabbit model study, 0.1%, 0.2%, and 0.4% (PEG)-BHD1028 solutions were tested, and a 0.05% cyclosporine treatment group was included as a positive reference and comparison group. Compared to the vehicle group, the tear volume measured by Schirmer's tear test (STT) on Day 5 was significantly increased in all the other groups of 0.1% (p<0.001), 0.2% (p<0.05), and 0.4% (PEG)-BHD1028 (p<0.01) groups and the 0.05% cyclosporine treatment group (p<0.01) (see Table 2).

TABLE 2

| | Period after | | | | | |
|---|---|---|---|---|---|---|
| | inducing | | | | | |
| Rabbit | dry eye | | (PEG)-BHD1028 | | | |
| model | syndrome | Vehicle | 0.1% | 0.2% | 0.4% | Cyclosporine |
| STT-2 | Baseline | 9.83 ± 1.74 | 6.67 ± 0.96 | 8.50 ± 1.39 | 9.17 ± 1.23 | 8.33 ± 0.38 |
| (mm) | Day 0 (EDE) | 2.67 ± 1.10 | 3.00 ± 1.22 | 3.33 ± 1.28 | 3.33 ± 1.22 | 2.50 ± 1.10 |
| | Day 5 | 1.17 ± 0.50 | 4.67 ± 0.65 | 3.33 ± 0.51 | 4.33 ± 0.45 | 4.17 ± 0.37 |
| | Day 10 | 2.17 ± 0.64 | 6.50 ± 0.87 | 7.50 ± 0.99 | 6.67 ± 1.02 | 4.33 ± 0.45 |
| TBUT | Baseline | 48.33 ± 3.88 | 49.67 ± 3.17 | 49.83 ± 2.10 | 53.00 ± 2.29 | 52.00 ± 2.25 |
| (sec) | Day 0 (EDE) | 8.20 ± 0.51 | 8.50 ± 1.0 | 8.89 ± 1.34 | 8.22 ± 1.16 | 8.92 ± 1.3 |
| | Day 5 | 8.81 ± 1.20 | 10.64 ± 0.99 | 15.68 ± 1.58 | 19.88 ± 1.42 | 11.19 ± 1.17 |
| | Day 10 | 10.22 ± 1.97 | 17.88 ± 1.91 | 17.72 ± 1.74 | 22.71 ± 1.87 | 11.49 ± 2.23 |
| CFS | Baseline | 0.33 ± 0.19 | 0.00 ± 0.00 | 0.00 ± 0.00 | 0.00 ± 0.00 | 0.17 ± 0.15 |
| (score) | Day 0 (EDE) | 8.75 ± 0.46 | 6.67 ± 0.30 | 9.17 ± 0.37 | 8.00 ± 0.62 | 6.05 ± 1.20 |
| | Day 5 | 8.50 ± 0.89 | 7.17 ± 1.21 | 6.83 ± 0.64 | 5.67 ± 0.38 | 5.67 ± 0.81 |
| | Day 10 | 12.50 ± 1.24 | 6.67 ± 0.56 | 6.17 ± 0.55 | 3.83 ± 0.64 | 6.17 ± 1.04 |

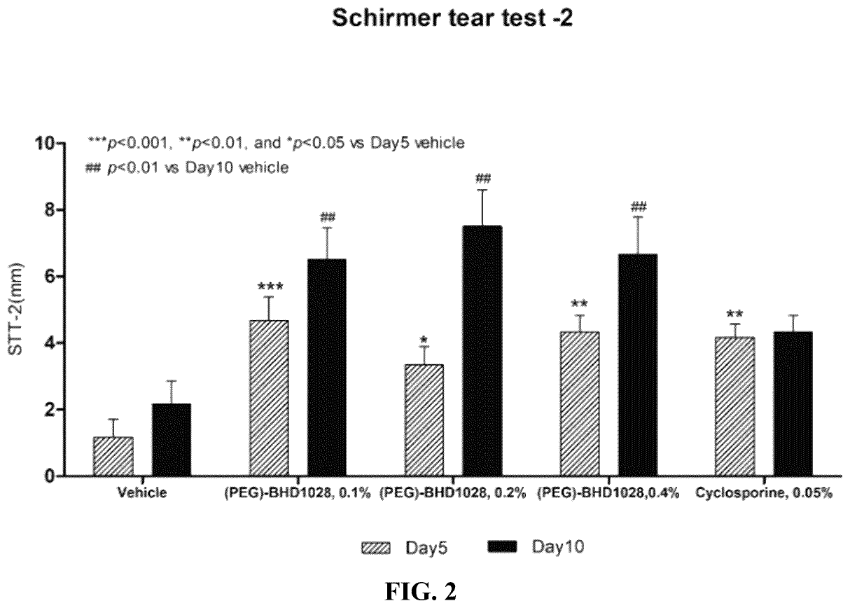

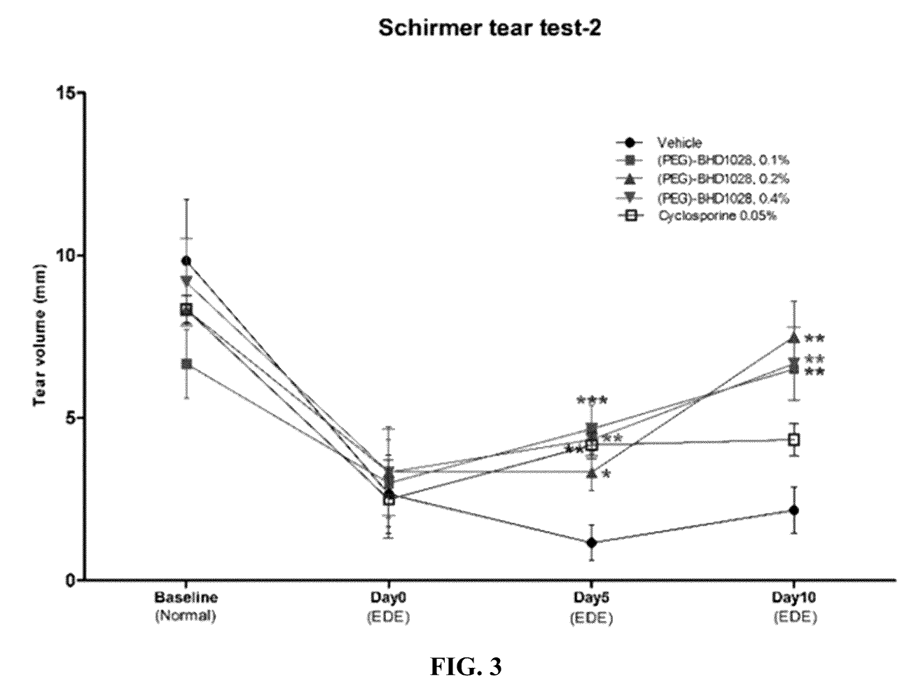

After 10 days, the tear production in all the (PEG)-BHD1028 groups was significantly increased (p<0.01) compared to the vehicle group (see Table 2 and FIGS. 2 and 3). However, the tear volume of the 0.05% cyclosporine group measured on Day 10 was not increase significantly compared to Day 5 or the vehicle group.

### Example 4: TBUT Evaluation

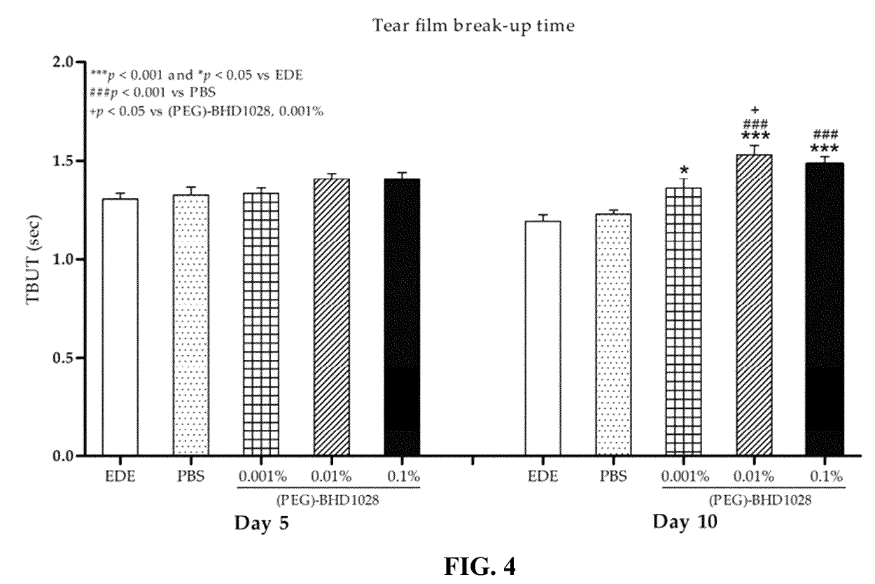

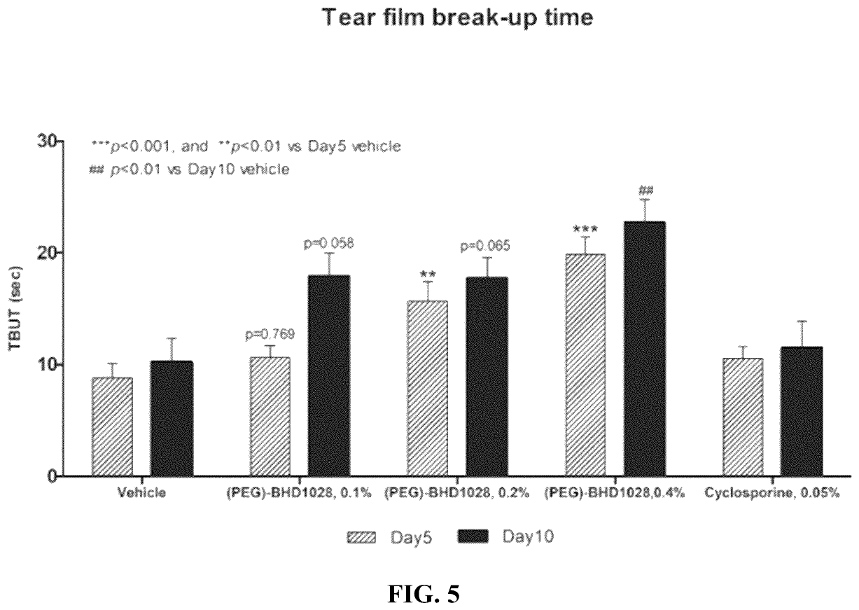

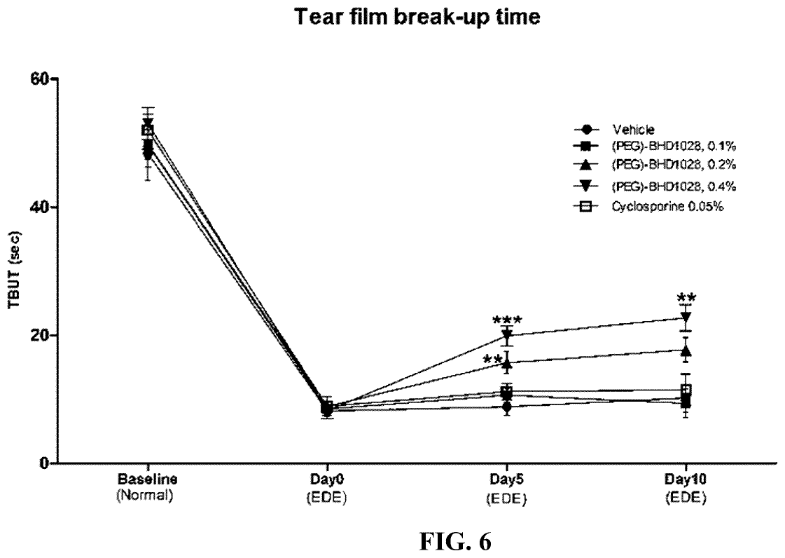

Tear film instability is an important pathological characteristic of DED and the most common indicator of tear quality. This was evaluated by measuring the time lapsed to tear film breakdown using a fluorescein dye, and the period was recorded as “tear film break-up time (TBUT).” In the EDE mouse model, the TBUT was significantly prolonged 10 days after the treatment in all the all (PEG)-BHD1028 groups at p<0.05 compared to the vehicle group (see Table 2 and FIG. 4). The improvement in the 0.01% (PEG)-BHD1028 group was greater than that in the 0.001% (PEG)-BHD1028 group on Day 10 (p<0.05), but there was no difference between 0.01% and 0.1%. In the EDE rabbit model, the TBUT of all the (PEG)-BHD1028 treatment groups was significantly greater than that of the vehicle group at each of p<0.01 and p<0.001 on Day 5 (see Table 2 and FIG. 5). After 10 days, the TBUT of the 0.4% (PEG)-BHD1028 group significantly increased compared to the vehicle group (p<0.01). Although not statistically significant, the 0.1% and 0.2% TBUT-treated groups exhibited improvement compared to the vehicle group or the 0.05% cyclosporine group after 10 days of the treatment (p<0.058 and p<0.065, respectively). The TBUT of the 0.2% and 0.4% (PEG)-BHD1028 groups tended to continuously increase over time, while the TBUT of the vehicle group or 0.05% cyclosporine group tended to plateau (see FIG. 6).

### Example 5. CFS Score Evaluation

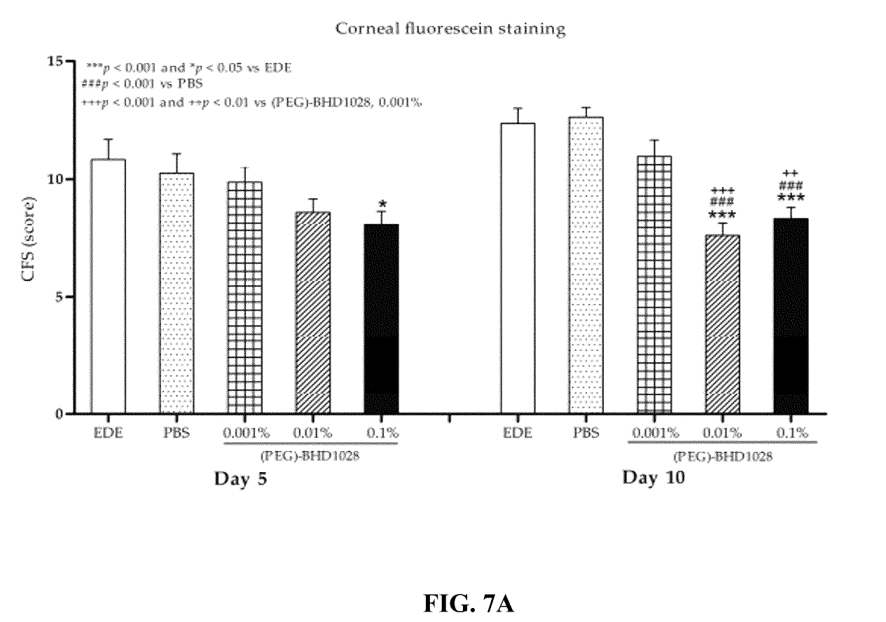

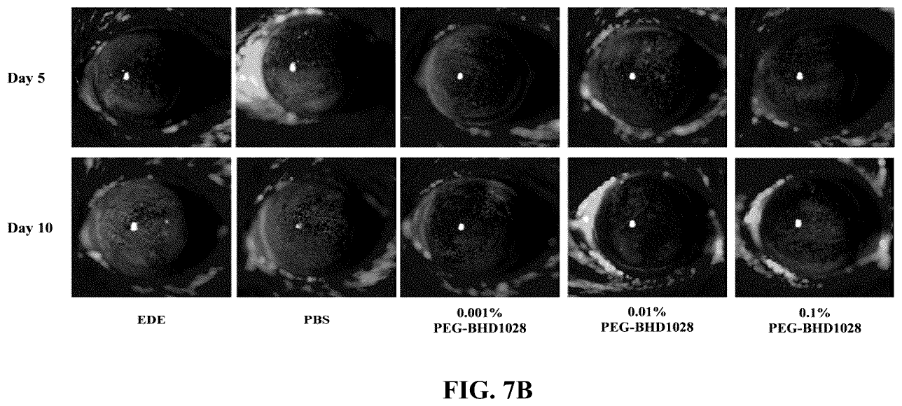

The clinical severity of the cornea was evaluated using a corneal fluorescein staining (CFS) method, which is widely used to detect foreign substances and corneal surface damage. A higher CFS score indicates more severe corneal epitheliopathy. As can be seen in Table 1 and FIGS. 7A-7B, the average CFS score of the 0.1% (PEG)-BHD1028 treated mouse group was significantly lower than that of the EDE group after 5 days (p<0.05). After 10 days of the treatment, the scores of 0.01% and 0.1% (PEG)-BHD1028 treated animals were significantly lower than that of the EDE animals.

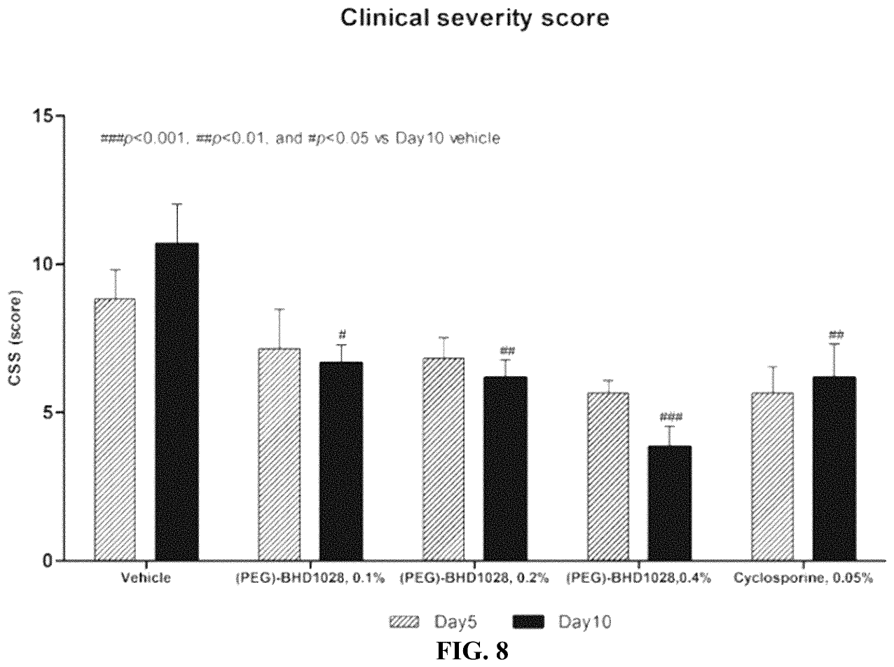

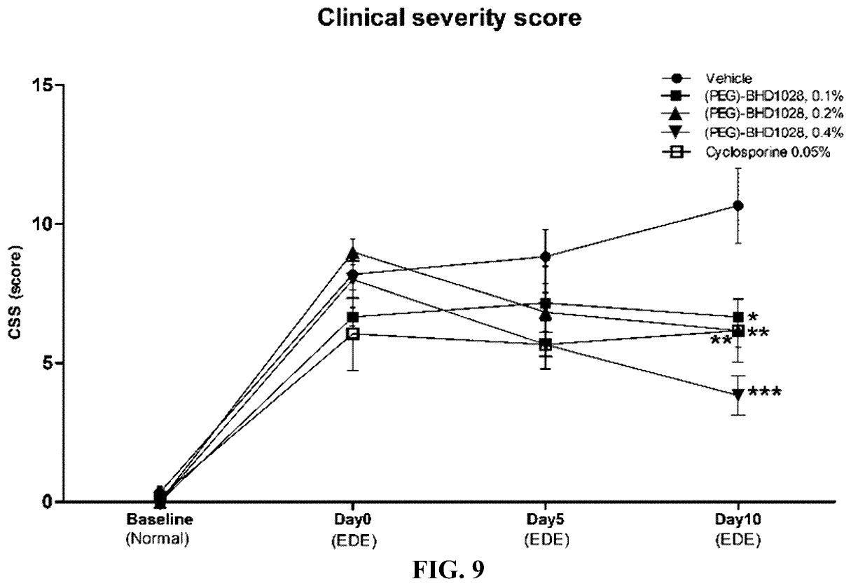

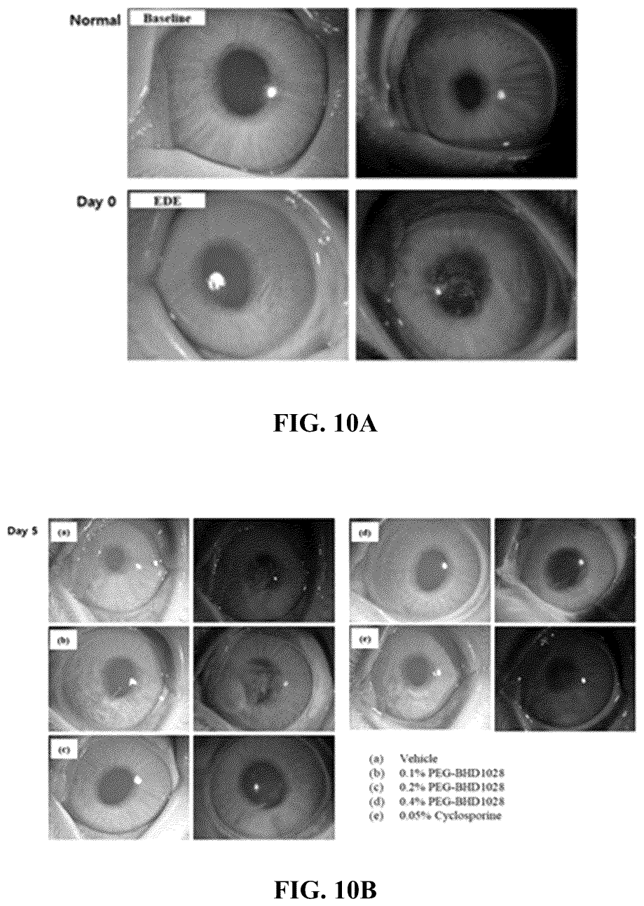

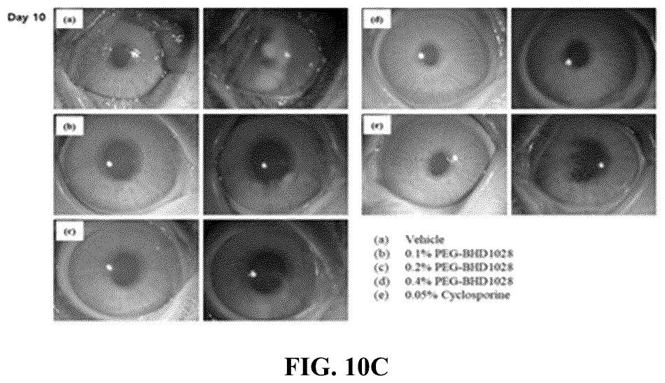

In the EDE rabbit model, there was no statistically significant difference between the all the (PEG)-BHD1028 groups or the 0.05% cyclosporine treatment group and the vehicle group after 5 days of the treatment. However, after 10 days, the CFS score were significantly reduced in a dose-dependent manner in all the (PEG)-BHD1028 groups and in the cyclosporine 0.05% treated animals (p<0.01) compared to the vehicle group (see Table 2, FIGS. 8 and 10). In addition, the dynamic slope of the CFS score change from Day 1 to Day 10 in the 0.2% and 0.4% (PEG)-BHD1028 treatment groups was −0.42 and −0.33, respectively, while the slope of the 0.05% cyclosporine group was +0.013. These results mean that the therapeutic effect of 0.2% and 0.4% (PEG)-BHD1028 on improving the clinical severity is better than that of 0.05% cyclosporine (see FIGS. 9 and 10).

### Example 6. Confirmation of Anti-Inflammatory Effect

In DED, corneal barrier destruction and epithelial cell loss are accompanied by bone marrow and T cell infiltration. Activated T lymphocytes are the primary producers of Th-1 cytokines and interferon (IFN)-γ, which promote cell death at the ocular surface. To evaluate the anti-inflammatory effect of (PEG)-BHD1028 in the EDE mouse model, immune cells that play the role of inflammatory cell surface markers were quantified by flow cytometry with one animal per group.

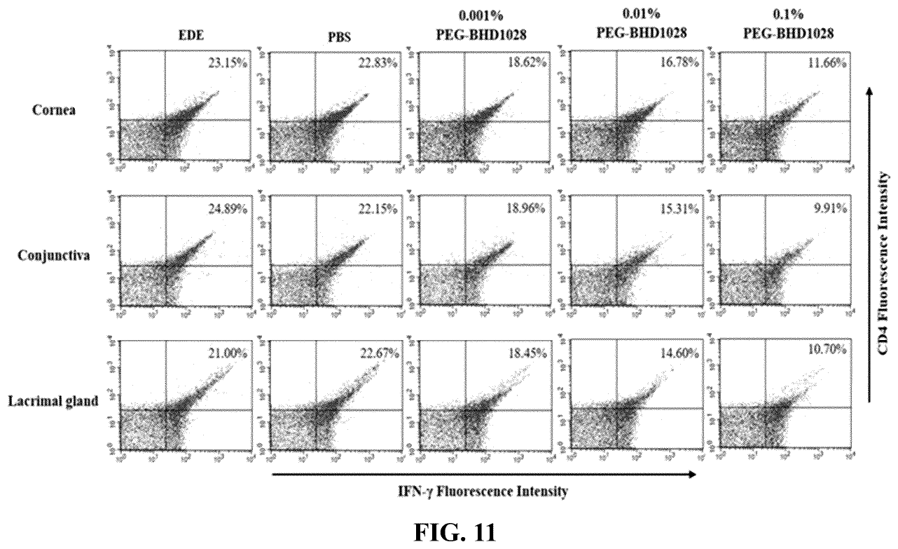

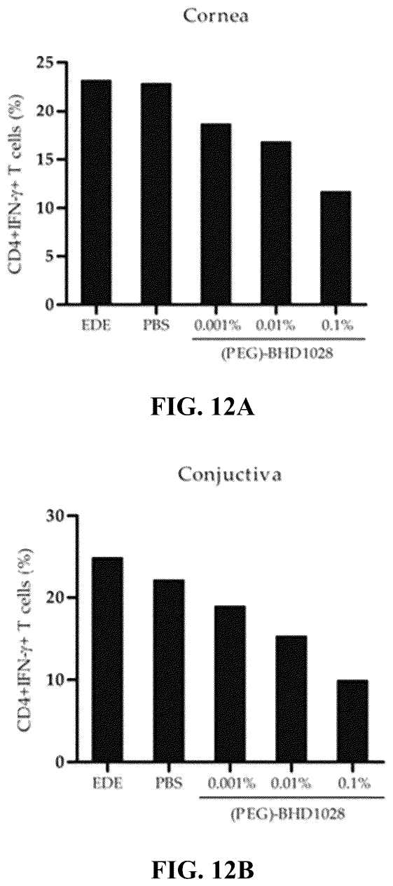

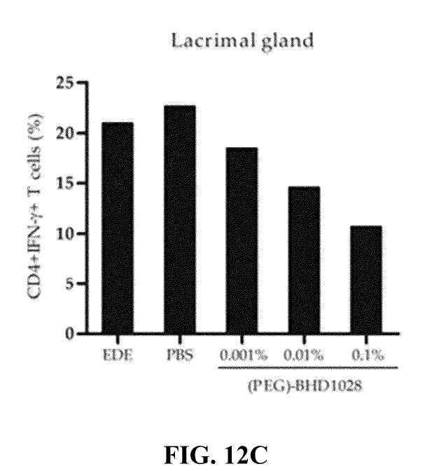

The CD4+IFN-γ+T cell density in the cornea of the EDE group and the vehicle groups was 23.15% and 22.83%, respectively, while it was 18.62% and 16.78%, and 11.66%, in the 0.001%, 0.01%, and 0.1% (PEG)-BHD1028 groups, respectively. The CD4+IFN-γ+T cell density in the conjunctiva of the EDE group and the vehicle group was 24.89% and 22.15%, respectively, and it was 18.96%, 15.31%, and 9.91% in the 0.001%, 0.01%, and 0.1% (PEG)-BHD1028 groups, respectively. The CD4+IFN-γ+T cell density in the lacrimal gland of the EDE group and the vehicle group was 21.00% and 22.67%, respectively, and the immune cell was 18.45% and 14.60%, and 10.70% in the 0.001%, 0.01%, and 0.1% (PEG)-BHD1028 group, respectively (see FIGS. 11 and 12).

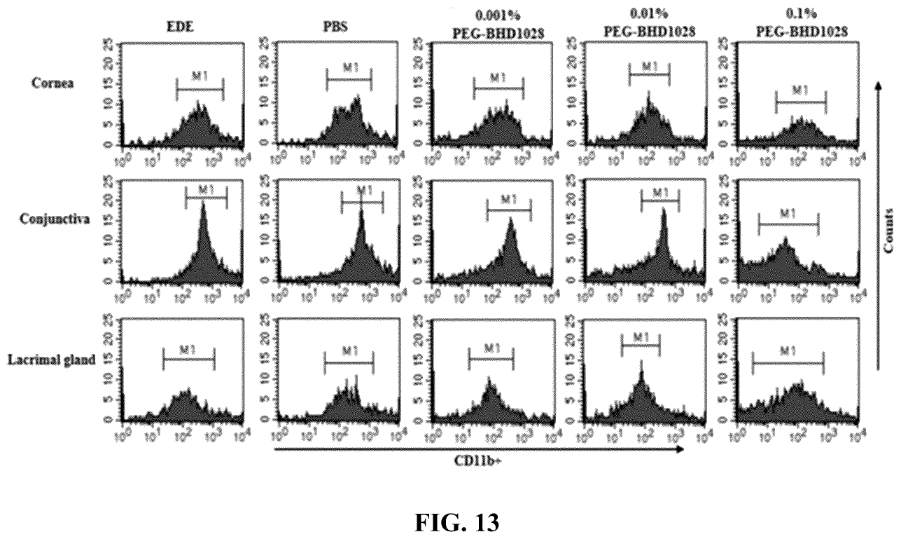

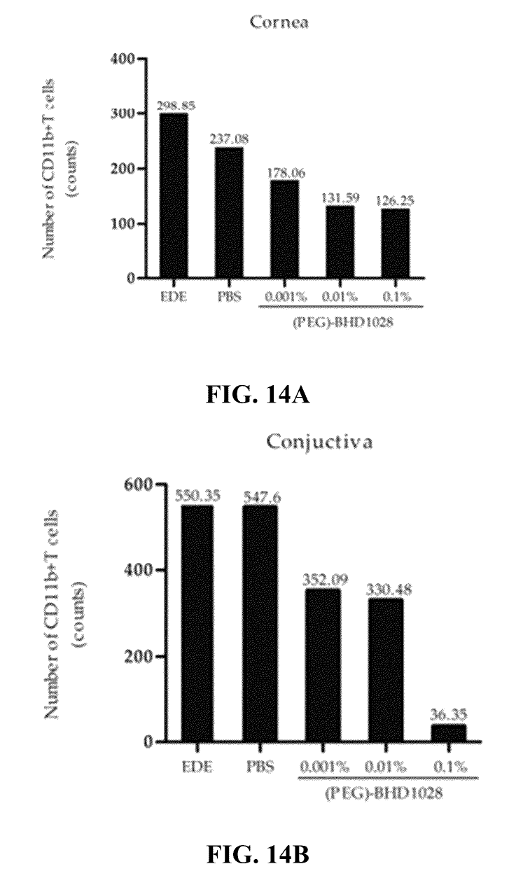

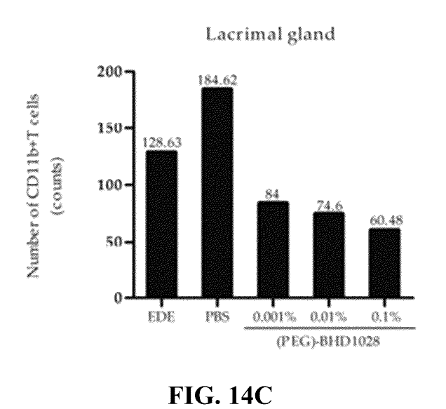

The geometric mean number of CD11b+ monocytes in the cornea of the EDE group and the vehicle group was 298.85 and 237.08, respectively. The average number in the 0.001%, 0.01%, and 0.1% (PEG)-BHD1028 groups was 178.06, 131.59, and 126.25, respectively. The number of CD11b+ monocytes in the conjunctiva of the EDE group and the vehicle group was 550.33 and 547.60, respectively, and it was 352.09, 330.48, and 36.35 in the 0.001%, 0.01%, and 0.1% (PEG)-BHD1028 treatment groups, respectively. In addition, the number of CD11b+ monocytes in the lacrimal gland of the EDE group and the vehicle group was 128.63 and 184.62, respectively, and it was 84, 74.60, and 60.48 in the 0.001%, 0.01%, and 0.1% (PEG)-BHD1028 groups, respectively (see FIGS. 13 and 14). The results of the CD4+IFN-γ+T cell density and the CD11b+ cell counts in the cornea, conjunctiva and lacrimal gland are indicative of an effective anti-inflammatory effect of (PEG)-BHD1028.

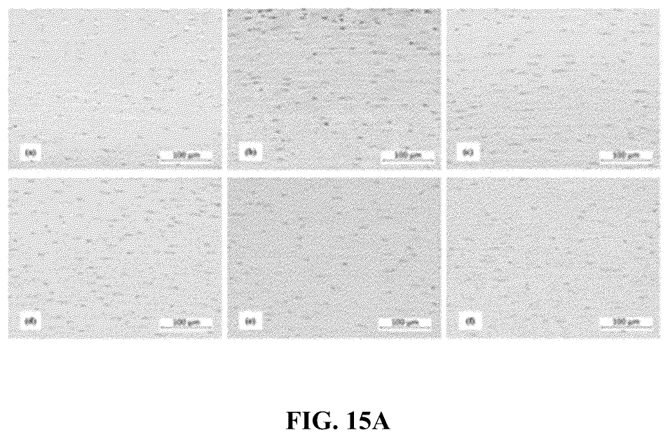

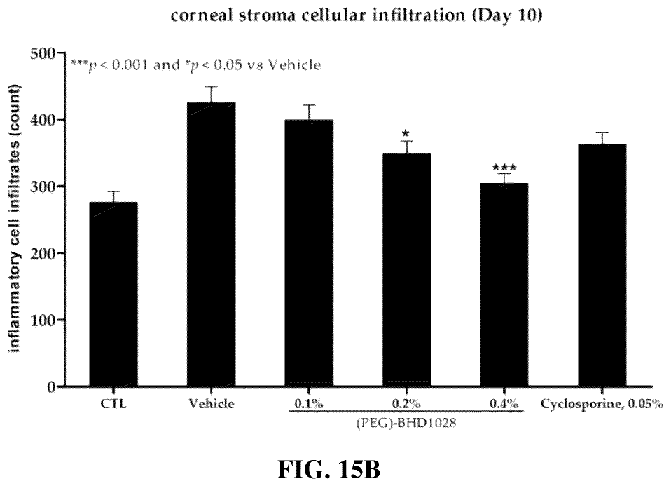

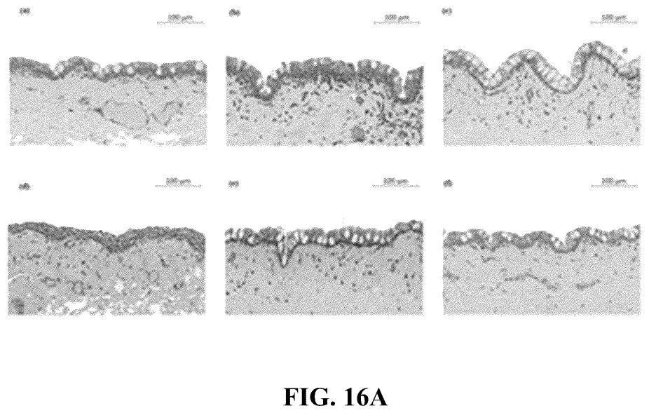

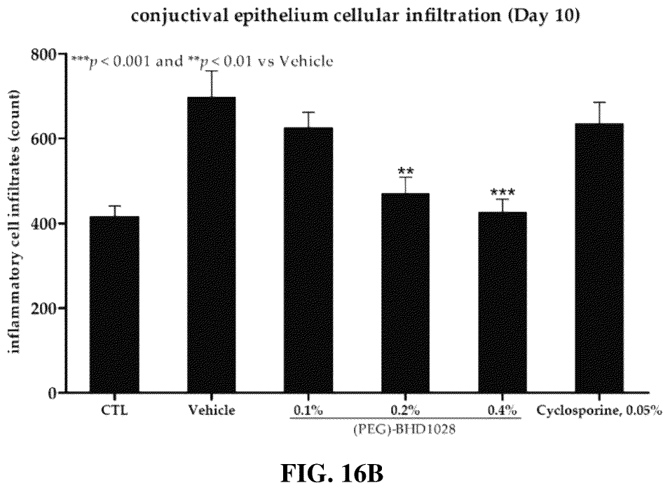

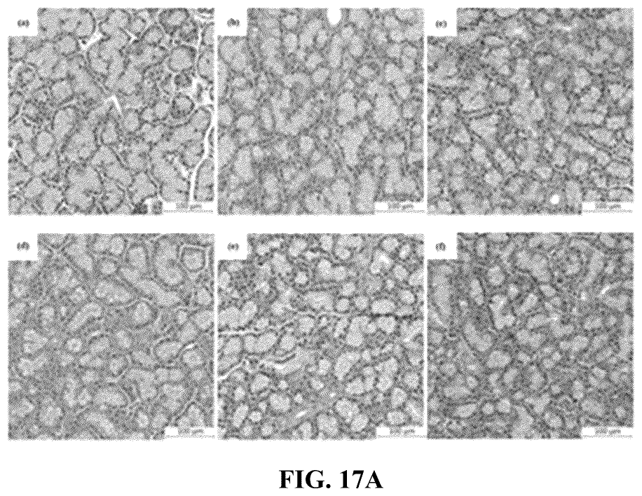

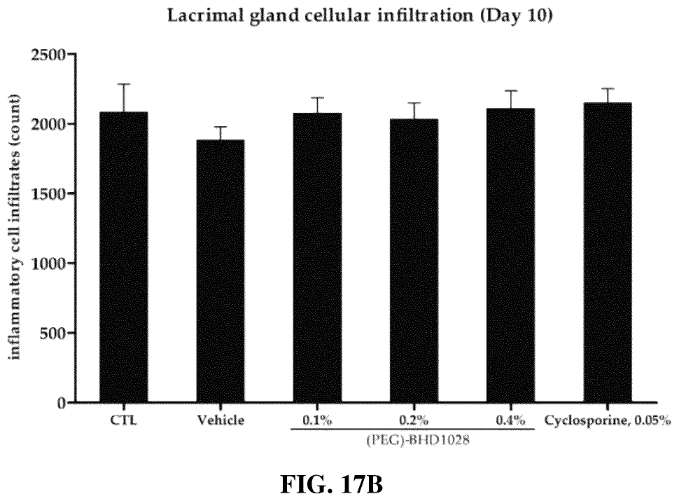

The DED inflammatory pathway also includes the infiltration of differentiated (antigen-specific) T cells into the cornea, conjunctiva, and lacrimal gland. To evaluate the anti-inflammatory effect of (PEG)-BHD1028 in the EDE rabbit model, the number of inflammatory cells infiltrated into the stromal layer was measured by H&E staining 10 days after the treatment. The range of the immune cell counts in the corneal stroma of six eyes of the normal control group was 273±16.08 in the normal control group, 424.78±22.64 in the vehicle group, 399.39±22.22 in the 0.1% (PEG)-BHD1028 group, 349.89±17.18 in the 0.2% (PEG)-BHD1028 group, 305.06±13.88 in the 0.4% (PEG)-BHD1028 group, and 363.07±17.75 in the 0.05% cyclosporine group (see FIG. 15). The number of immune cells in the corneal stroma of the 0.2% and 0.4% (PEG)-BHD1028 groups was significantly lower than that of the vehicle group at p<0.05 and p<0.001, respectively. However, there was no significant difference between the 0.05% cyclosporine group and the vehicle group or 0.1% (PEG)-BHD1028 group. The number of immune cells infiltrated into the conjunctiva was 415.67±25.01 in the normal control group and 698.78±60.51 in the vehicle-treated group, and it was 624.94±29.29, 472.41±39.06, and 427.22±29.29 in the 0.1%, 0.2%, and 0.4% (PEG)-BHD1028 treatment groups, respectively. The number of the infiltrated immune cells in the cyclosporine 0.05% group was 635.39±50.70 (see FIG. 16). The immune cells were significantly less in the conjunctival epithelium of the 0.2% and 0.4% test groups than in the vehicle-treated group (p<0.001). However, there was no statistically significant difference between the 0.05% cyclosporine group and the vehicle group. There was no difference between the study groups in the lacrimal gland (see FIGS. 17A-17B).

### Example 7: Evaluation of Corneal Epithelial Thickness in EDE Rabbit Model

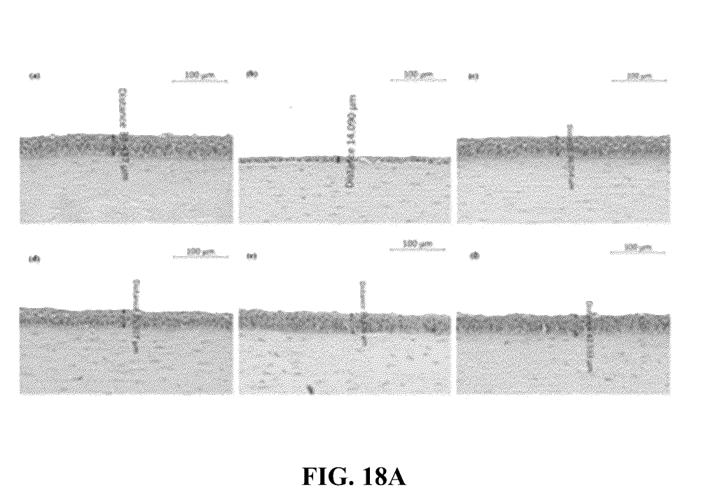

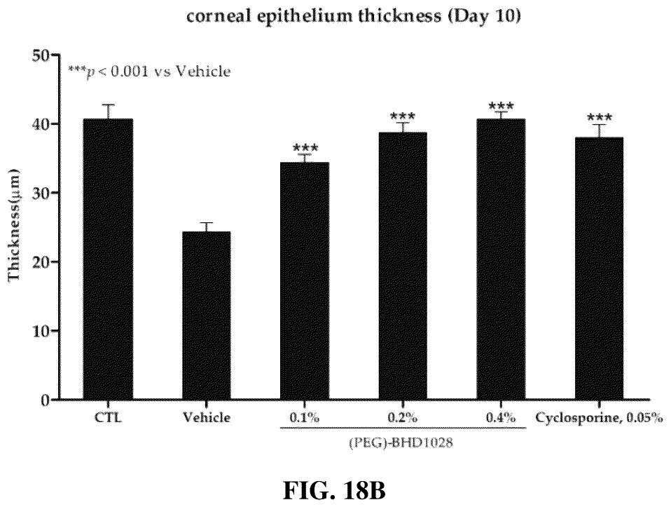

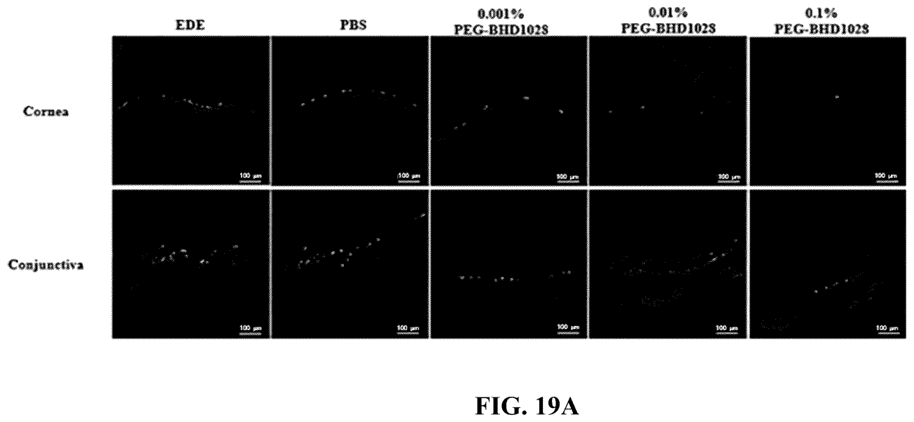

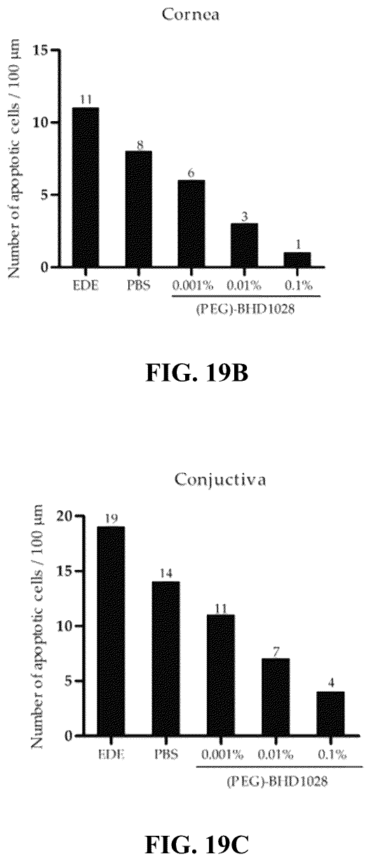

The pathophysiology of dry eye syndrome includes pathological changes in the epithelium such as erosion, cicatrix, scar, corneal neovascularization and, in severe conditions, corneal perforation. The protective effect of (PEG)-BHD1028 on the epithelium was evaluated by measuring the corneal thickness using ZEN (Zeiss, Germany) microscopy software program after H&E tissue staining. In six eyes, the mean corneal epithelial thickness of all the (PEG)-BHD1028 treatment groups and the 0.05% cyclosporine treatment group was significantly greater than that of the vehicle treated animals at p<0.001 (see FIGS. 18A-18B). However, there was no significant difference between the (PEG)-BHD1028 treatment groups and the 0.05% cyclosporine treatment group (see FIGS. 18A-18B).

### Example 8: Antiapoptotic Effect of (PEG)-BHD1028 in EDE Mouse and Rabbit Models

As DED progresses, the epithelium of the anterior segment of the eye undergoes pathological deformations, including functional damages and structural changes such as abnormal tear sebum secretion and pathogenic cell death. Apoptotic cells that were present in an equal area of the cornea and conjunctiva of one eye per group were quantified by the TUNEL assay. As shown in FIG. 8, (PEG)-BHD1028 exhibited a dose-dependent antiapoptotic effect in the cornea and conjunctiva of the mice.

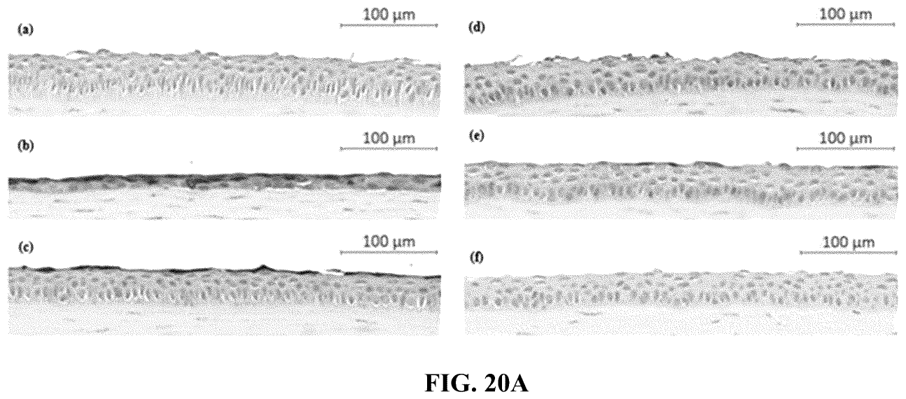

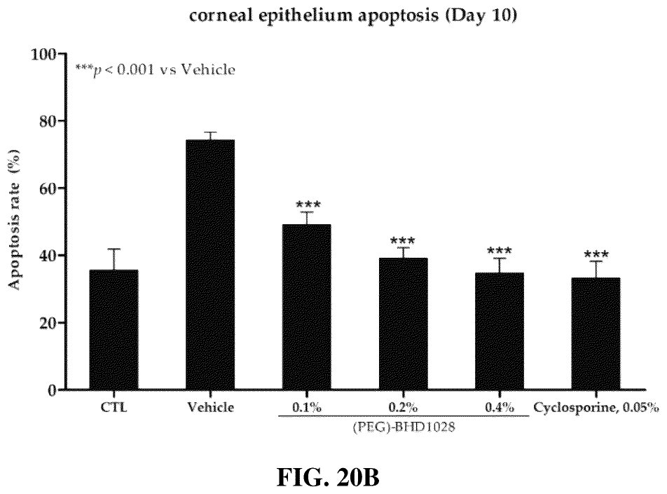

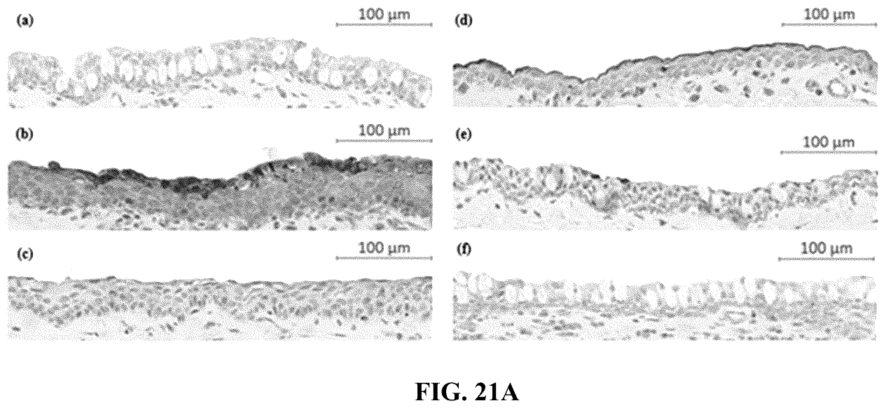

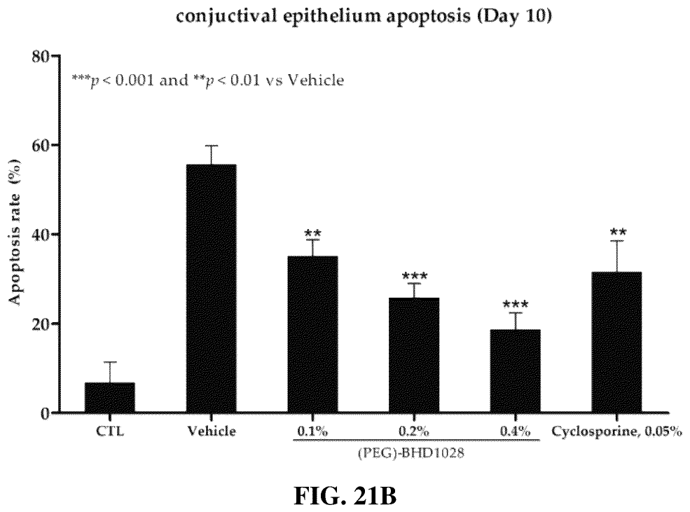

The percentage of the apoptotic cells per unit area was evaluated after 10 days in the rabbit model. In the cornea, apoptotic cells were detected at a percentage of 35±6% in the normal control group and 74±2% in the vehicle group. In the 0.1%, 0.2%, and 0.4% (PEG)-BHD1028 treatment groups, the percentage was 40±12%, 39±3%, and 35±4%, respectively. The percentage in the cyclosporine 0.05% group was 33±5%. The percentage of apoptotic cells in the cornea of all the (PEG)-BHD1028 groups and the 0.05% cyclosporine group was significantly lower than that of the vehicle group at p<0.001 (see FIGS. 20A-20B). In addition, the cell death rate in the conjunctiva was 7±5% in the normal control group and 56±4% in the vehicle group. The percentage in the 0.1%, 0.2% and 0.4% (PEG)-BHD1028 treatment groups was 35±4%, 26±3% and 19±4% on Day 10. The percentage of the 0.05% cyclosporine group was 32±7% (see FIGS. 21A-21B). All the tested (PEG)-BHD1028 concentrations and 0.05% cyclosporine significantly reduced corneal and conjunctival apoptosis compared to the vehicle group after 10 days of the treatment.

### Example 9: Comparative Analysis of 0.4% (PEG)-BHD1028 and 0.05%

### Cyclosporine in Rabbit EDE Model on Day 10

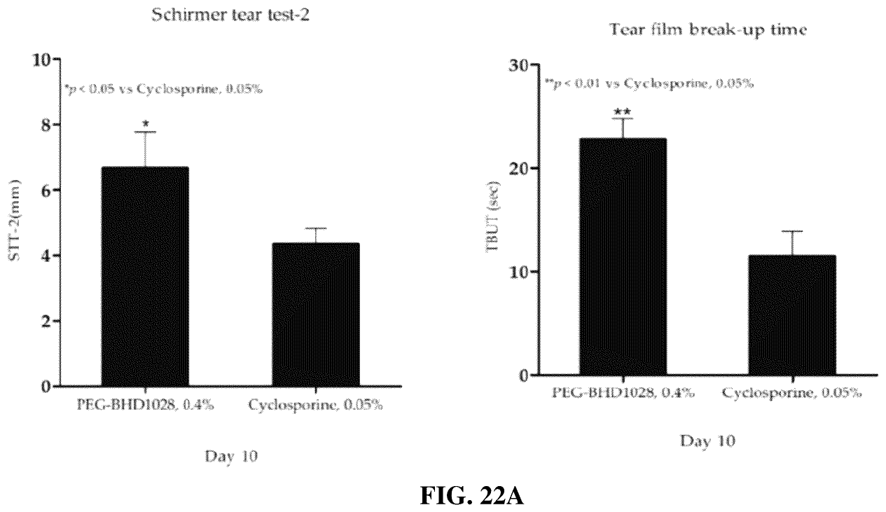

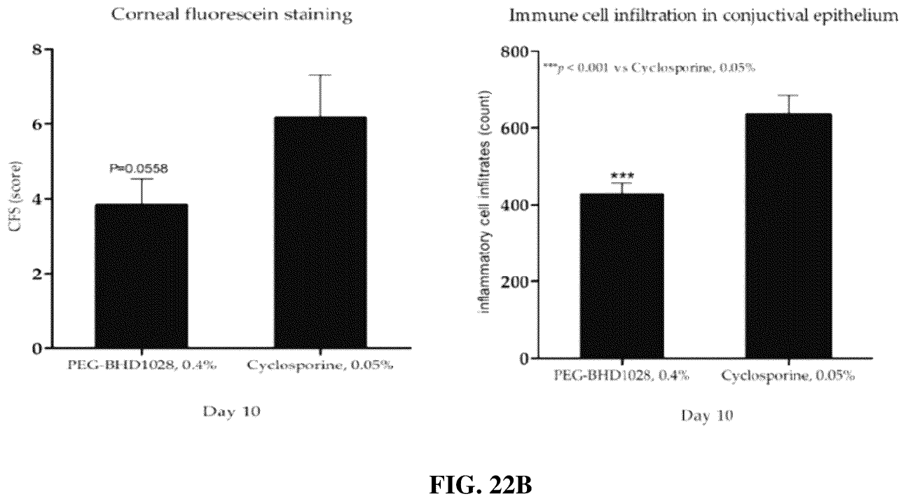

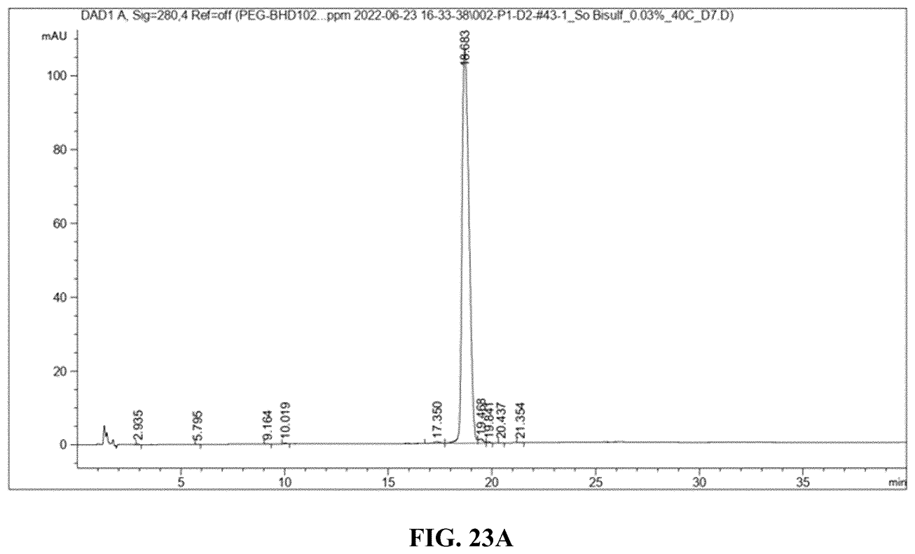

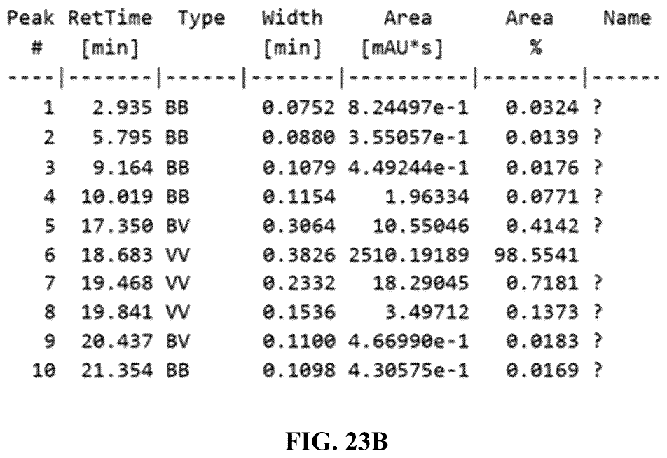

Based on the consistent therapeutic effect of 0.4% (PEG)-BHD1028, all the test results of 0.4% (PEG)-BHD1028 and 0.05% cyclosporine on Day 10 were comparatively analyzed using Student's t-test. The tear volume and TBUT of 0.4% the (PEG)-BHD1028 group were significantly greater than those of the 0.05% cyclosporine group (see FIGS. 22A-22B). There was no significant difference between the CFS score and the immune cell counts in the (PEG)-BHD1028 group and the 0.05% cyclosporine group. However, the mean CFS score of the 0.4% (PEG)-BHD1028 group was approximately 46% lower than that of the 0.05% cyclosporine treated animals (p=0.0558), and it tended to gradually improve over time. In terms of the immune cell counts, 0.4% (PEG)-BHD1028 showed a better effect in the conjunctival epithelium (p<0.001) (see FIGS. 23A-23B).

### Example 10: Preparation of Eye Drops Using (PEG)-BHD1028

Eye drops using (PEG)-BHD1028 were prepared by setting eye characteristics, pH, osmolarity, and viscosity as important parameters. To adjust the above parameters, various reagents and substances were added to the test formulation, which was stored at 40° C. to confirm the stability of the formulation. In addition, the reagents used in the target preparation were mainly determined by considering the stability of the drug and the suitability of the manufacturing process. The stability of the formulation was determined based on the RP-HPLC analysis data. The shelf life was set as the period before a single impurity reached 1.0 area %, reflecting the mean kinetic temperature (MKT).

### 10-1. Antioxidant

Antioxidants were screened to prevent the decomposition of the protein medicine, which is particularly vulnerable to oxidation. Two antioxidants were tested: sodium bisulfate and EDTA. As can be confirmed in FIGS. 23A-23B, in the case of the formulation including sodium bisulfate (0.03%), impurities rapidly increased on Day 7 at 40° C., and thus sodium bisulfate was excluded from the antioxidants.

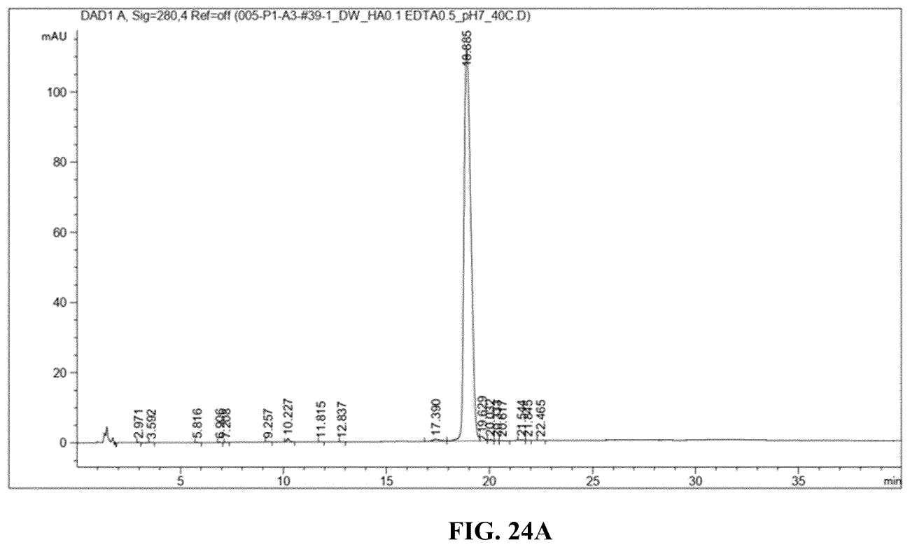

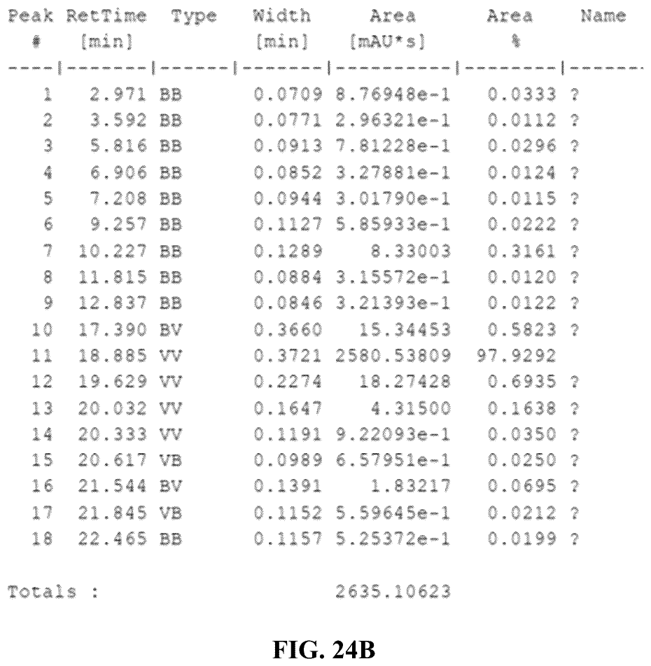

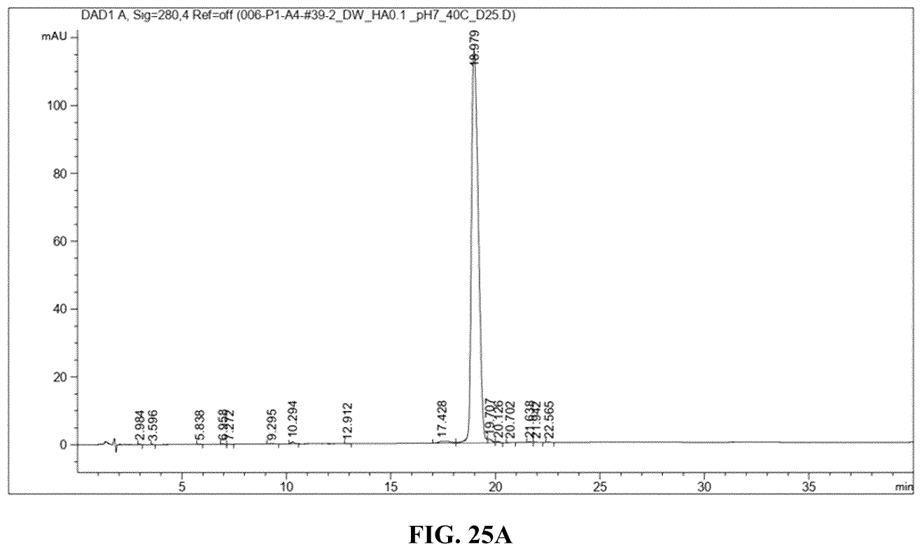

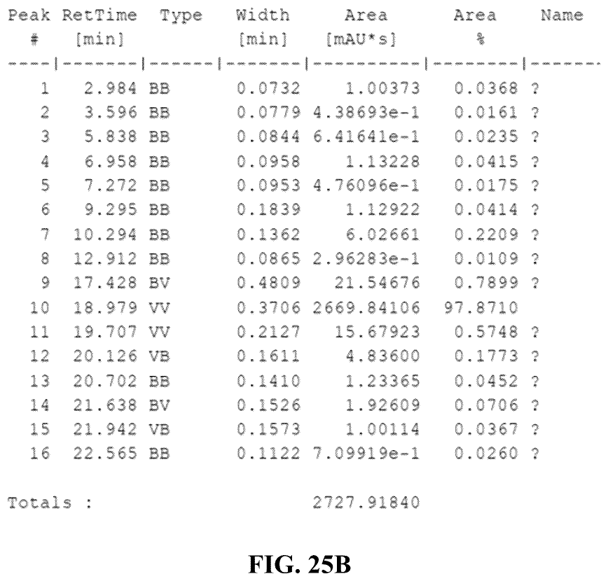

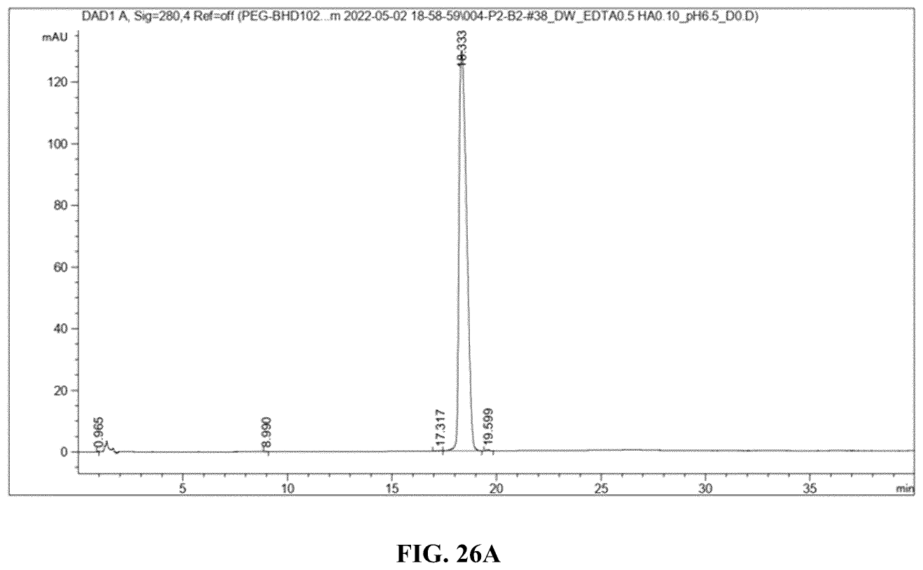

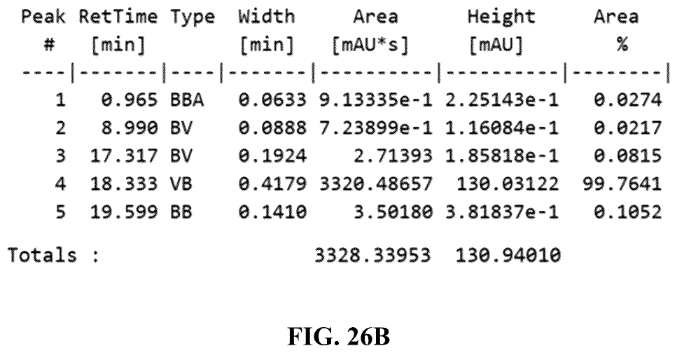

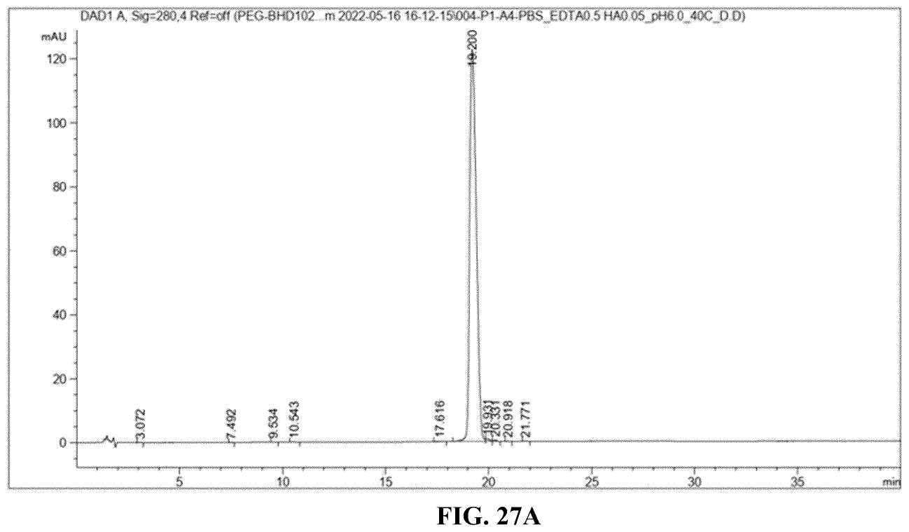

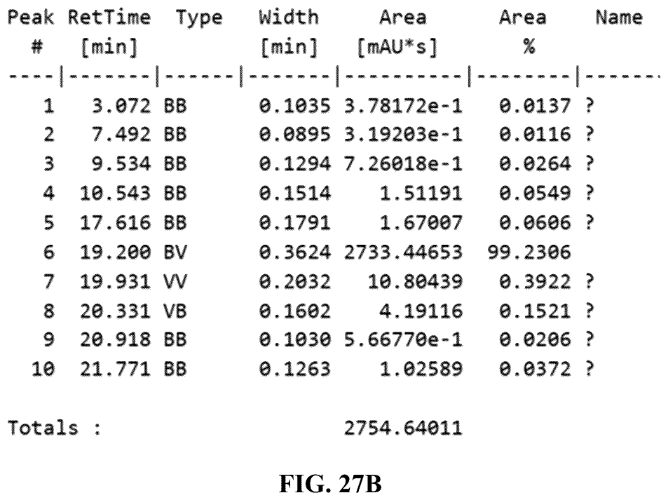

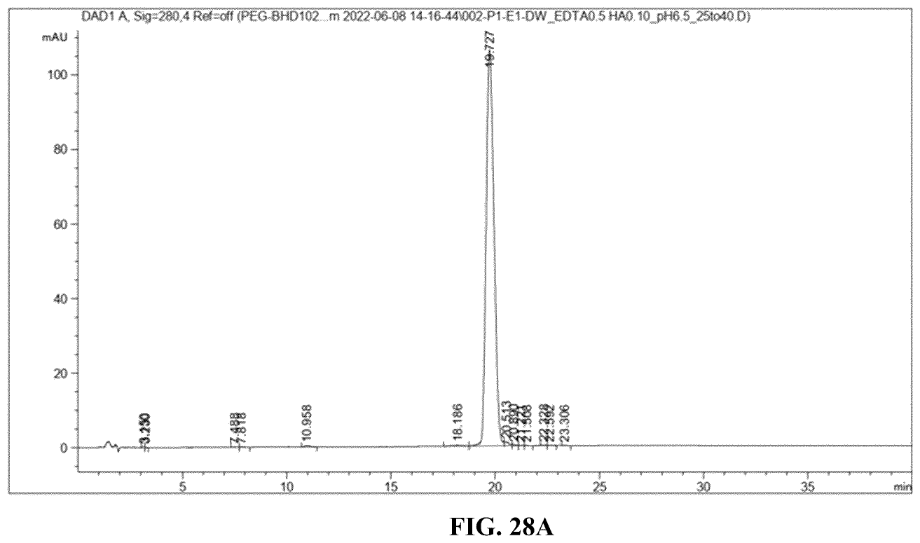

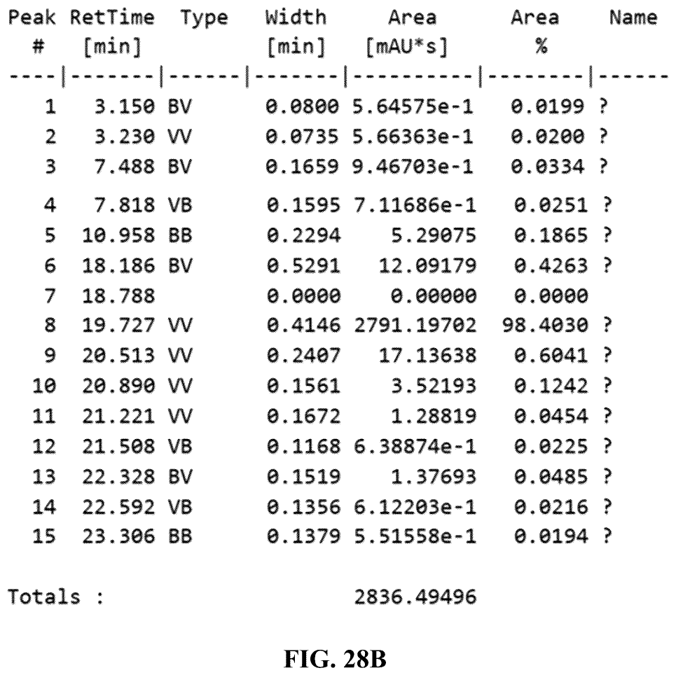

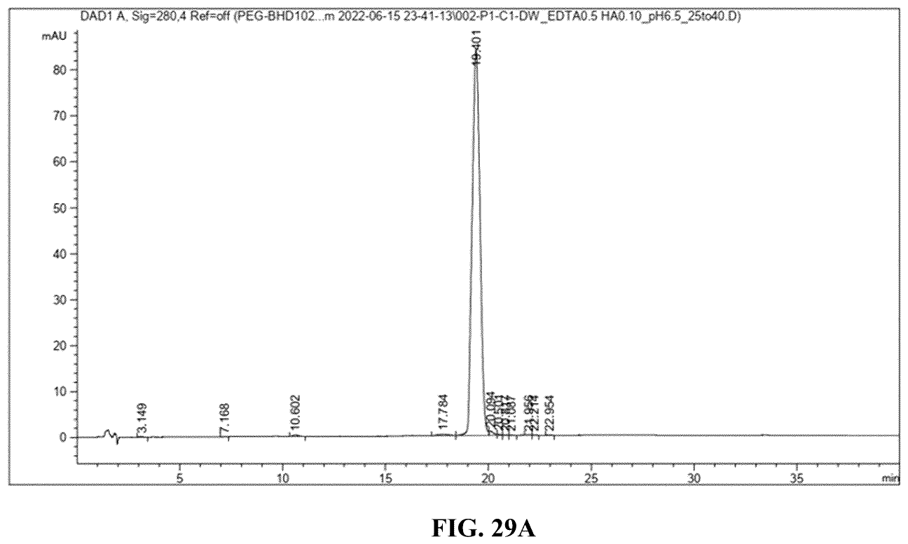

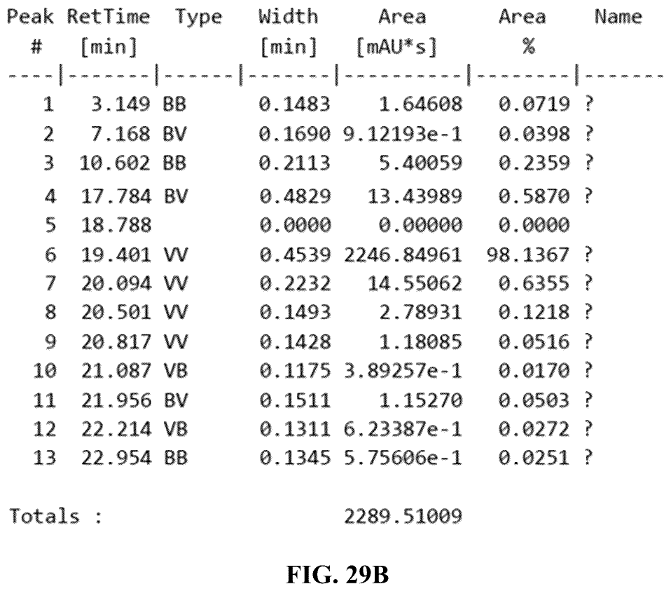

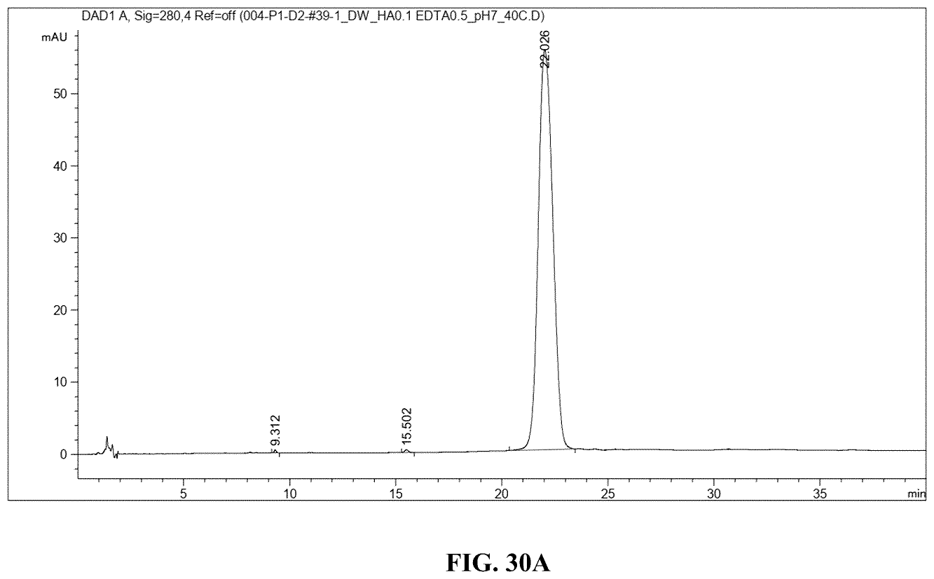

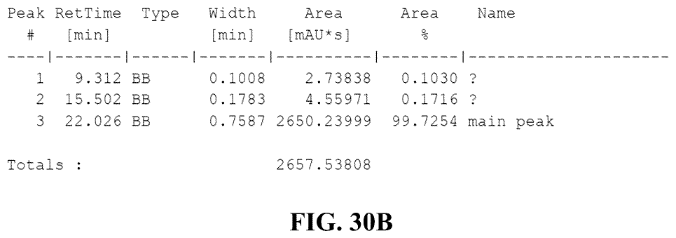

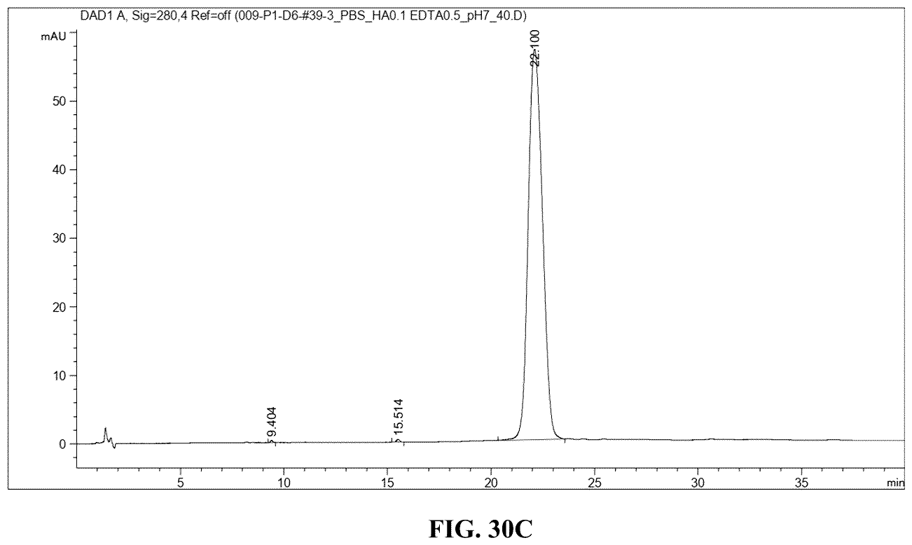

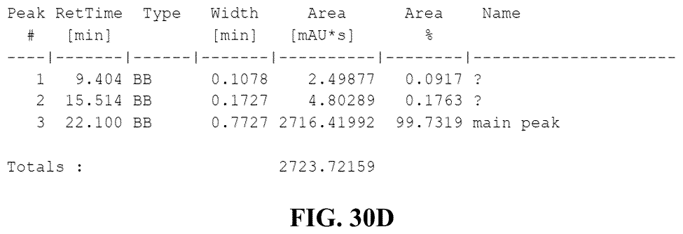

Two formulations were prepared to confirm the antioxidant effect of sodium EDTA: one including sodium EDTA (0.5%) and the other including no sodium EDTA. As can be confirmed in FIGS. 24 and 25, it was confirmed that stability was maintained even after storage at 40° C. for 25 days compared to the case when sodium bisulfate was added and storage at 40° C. for 7 days. In addition, as a result of a stability test according to the addition of EDTA, it was confirmed that the formulation including sodium EDTA was more stable than the formulation that did not. In addition, it was confirmed that EDTA sodium caused no particular sample decomposition. Therefore, EDTA sodium was set as the antioxidant.

### 10-2. Viscosity

Since a viscosity reagent is added in a solution form, a reagent that can be more easily prepared as a stock solution was selected as a viscosity reagent. The stock solution was prepared by adding a powder form to water and stirring until well dispersed. Methylcellulose may also be used as a viscosity reagent, but sodium hyaluronate is easier to prepare.

Eye drops including sodium hyaluronate at a concentration of 0.1% were prepared, and the results of a stability test showed that there were almost no impurities even when stored at 40° C. for 37 days (All impurities had less than 1.0 area %.). Based on the above information, sodium hyaluronate was set as the viscosity reagent. Meanwhile, as a result of evaluating the relationship between the content of (PEG)-BHD1028 and the viscosity using a rotational rheometer, as shown in Table 3, it was confirmed that as a result of containing 0.5% or 1.5% of (PEG)-BHD1028, the viscosity was similar to about 10 cP, which was the target viscosity between normal people and dry eye syndrome.

TABLE 3

| (PEG)-BHD1028 | | | |
|---|---|---|---|
| concentration | Unit | Shear rate | Test result |
| 0.5% | Pa · S | 100 (1/s) | 0.009 |
| 1.5% | Pa · S | 100 (1/s) | 0.010 |
| 4.5% | Pa · S | 100 (1/s) | 0.014 |
| Placebo | Pa · S | 100 (1/s) | 0.008 |

### 10-3. pH and Diluent

### Diluent

Water and PBS were tested as a diluent to maintain the target pH of the eye drops. However, there was no significant difference in stability data and pH between the two formulations.

pH

The normal pH range of tears is 6.5 to 7.6, while eye drops have a wider range than normal tears. Since the most stable pH for API within the pH range of normal tears is 6.5, the target pH of the eye drop formulation was set at 6.5. At pH 6.5, the API has cationic properties that may increase the retention time in the eye.

To evaluate the stability of eye drops according to pH, a 10 mM pH buffer [10 mM sodium phosphate buffer+0.02% sodium azide (pH 5.5, pH 6.0, pH 6.5, pH 7.0) or a 10 mM PBS+WELGENE fresh media 10×PBS (pH 7.4)] was mixed with (PEG)-BHD1028 and the resulting mixture was incubated at 40±2° C., and then the stability was evaluated through an HPLC analysis. As a result of the evaluation, there was no significant difference in the residual amount between the formulation prepared at pH 6.5, which was set as the target pH of the eye drops, and the formulation prepared at 7.0, which was the average pH of the eyes, as shown in Table 4 below.

TABLE 4

| Residual | | | | |
|---|---|---|---|---|
| amount (%) | Day 0 | Day 1 | Day 3 | Day 7 |
| pH 5.5 | 100.0 | 98.4 | 97.2 | 96.6 |
| pH 6.0 | 100.0 | 99.8 | 99.0 | 98.1 |
| pH 6.5 | 100.0 | 99.7 | 98.7 | 97.3 |
| pH 7.0 | 100.0 | 99.2 | 98.7 | 94.9 |
| PBS (pH 7.4) | 100.0 | 97.1 | 92.3 | 89.9 |

When the pH of the formulation is lower than the pI value of the API (approximately 6.6), the API has a cationic nature, which may increase the retention time in the eye. Based on the above results, pH 6.5 was set as a parameter for the eye drop formulation in consideration of the effectiveness and economic feasibility.

### 10-4. Osmotic Pressure

Various reagents were tested to adjust the osmolarity of the eye drops to about 300 mOsm/kg, which was similar to the osmolality of the eyes, and the most stable reagent was selected as the osmolality reagent.

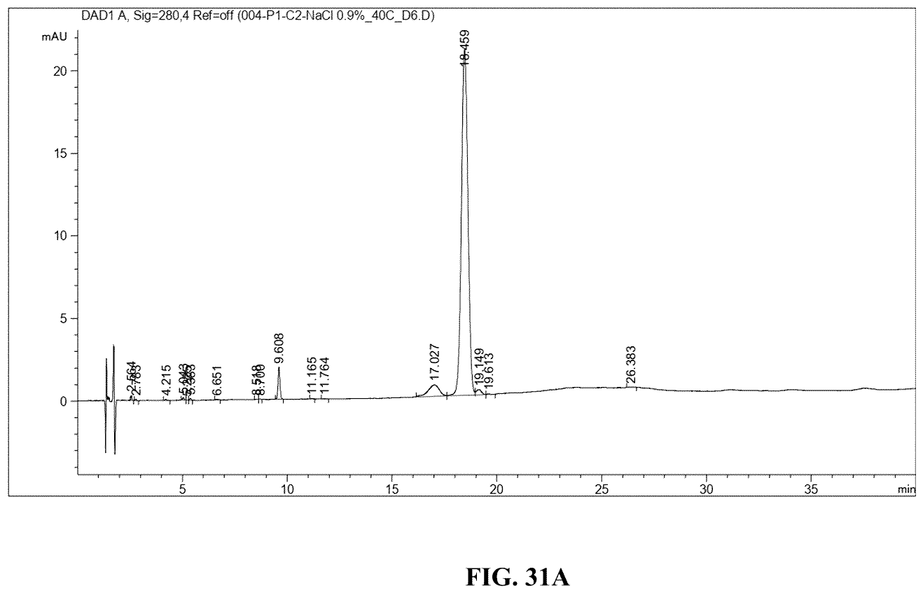

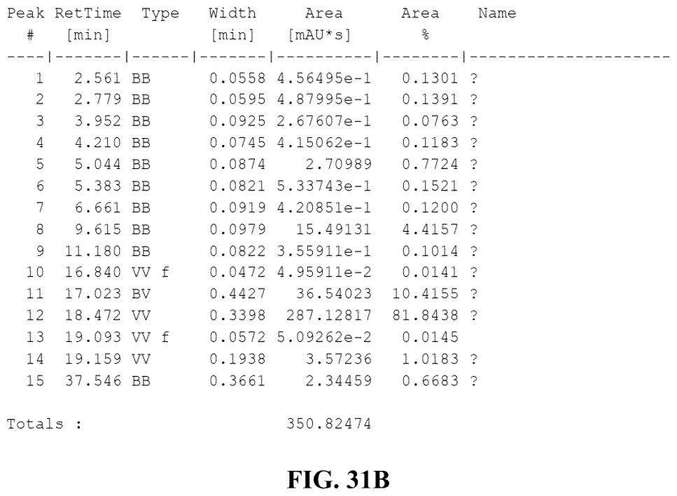

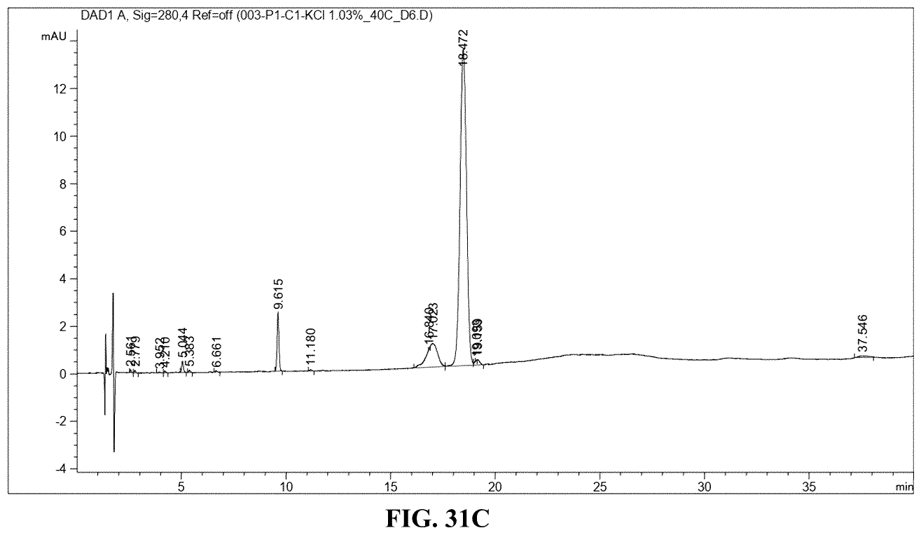

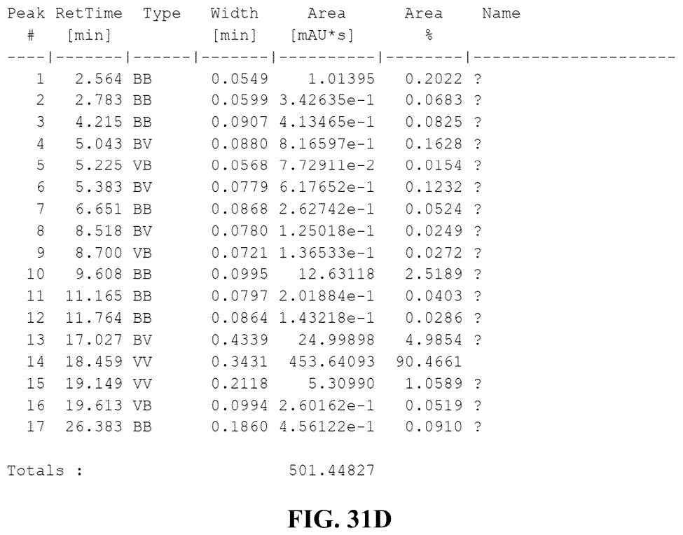

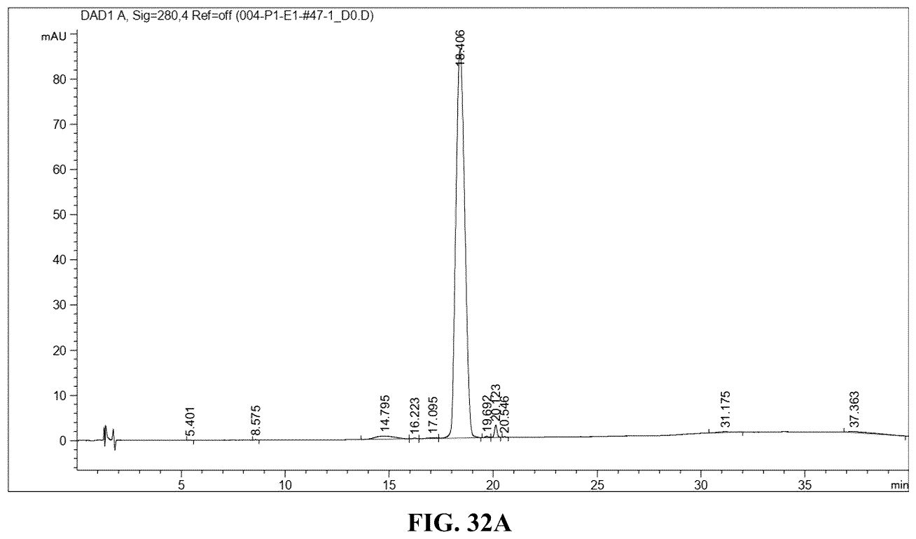

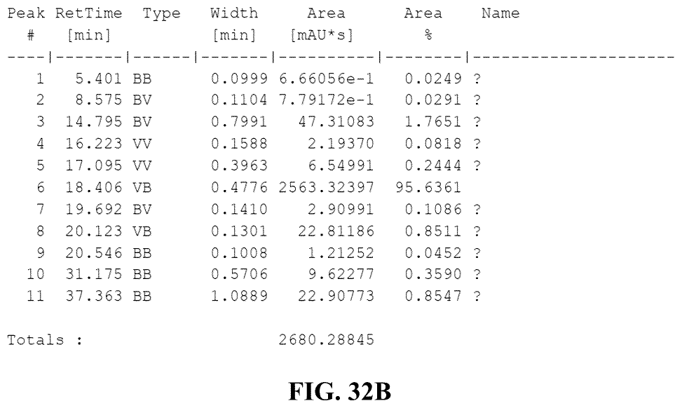

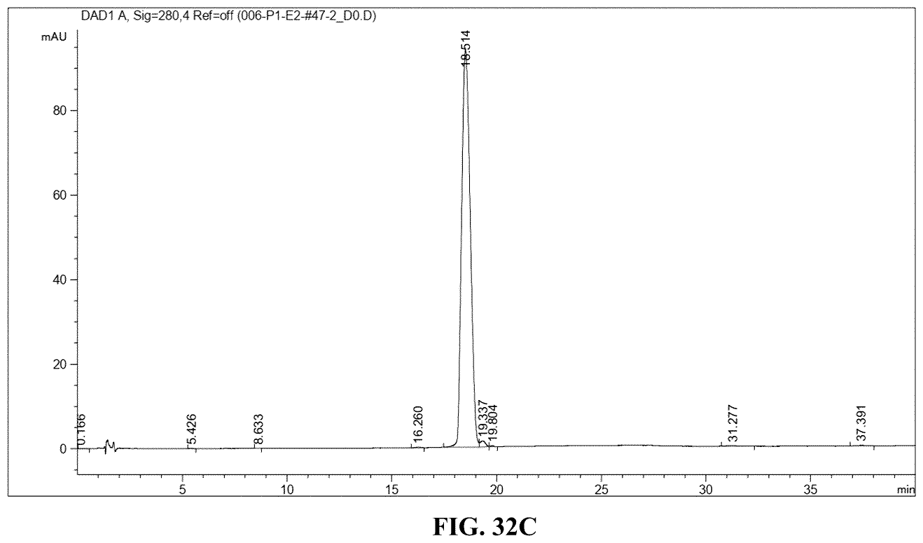

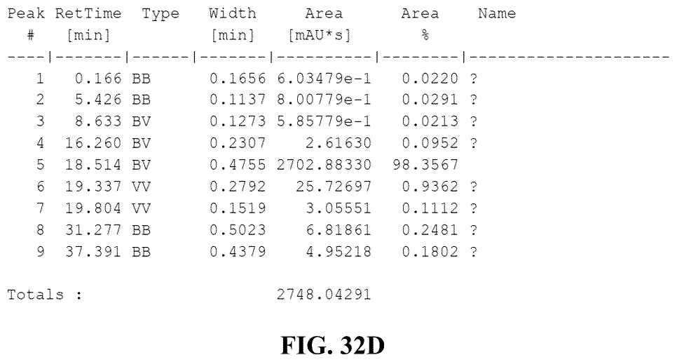

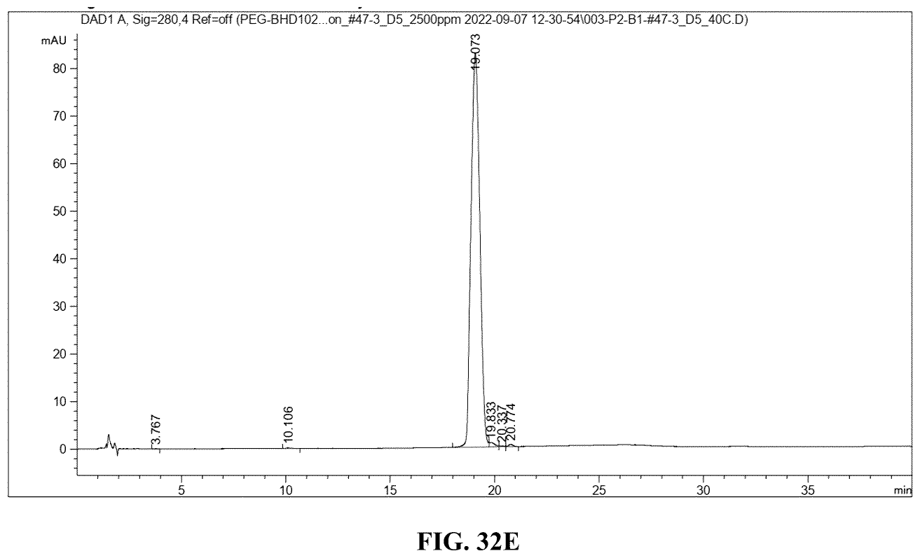

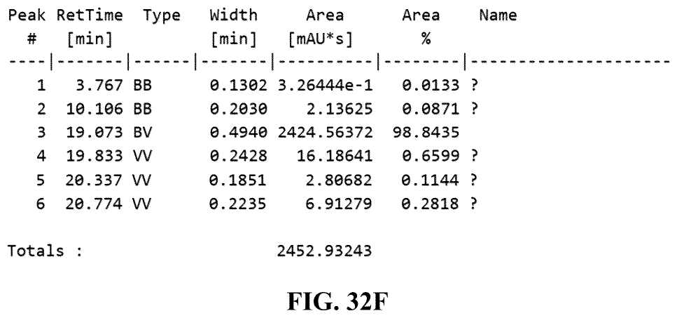

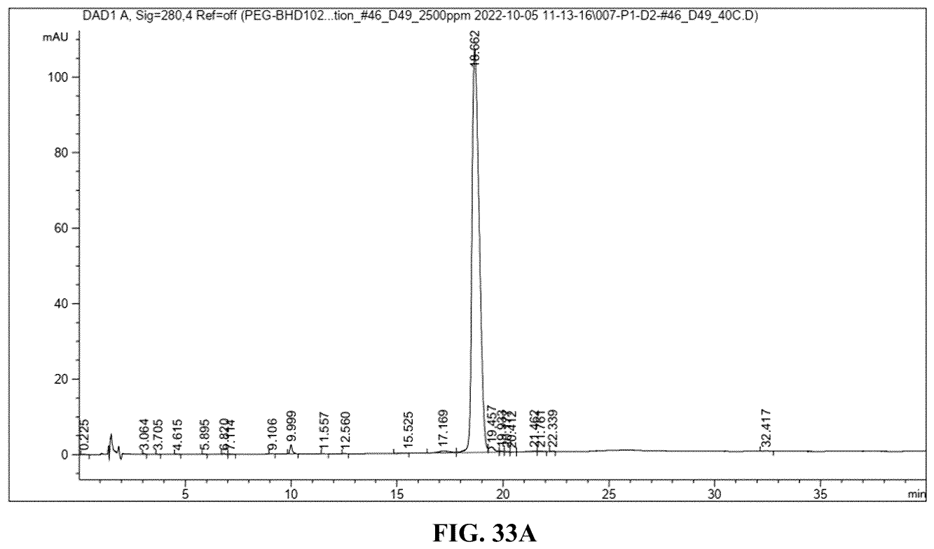

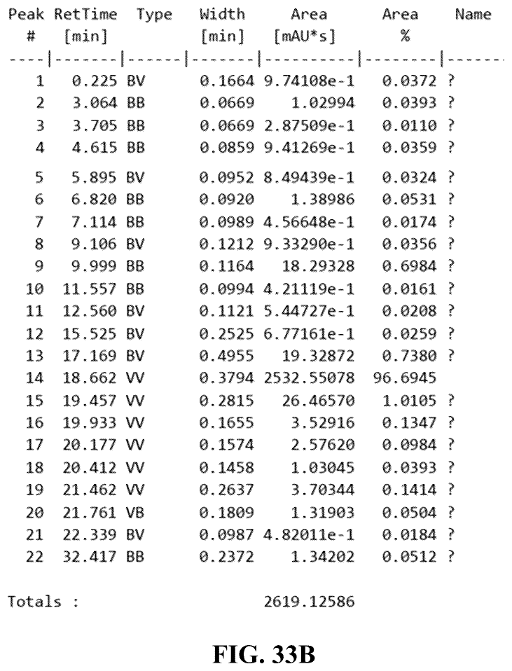

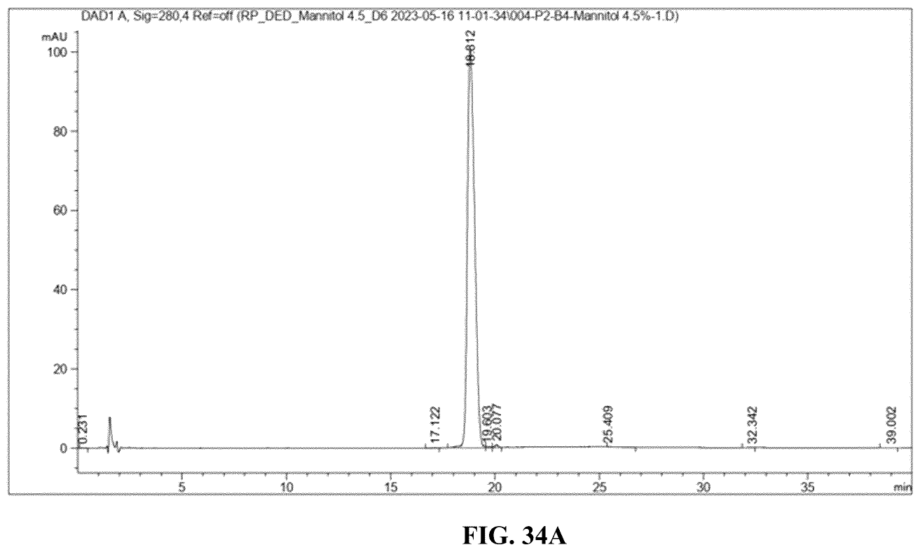

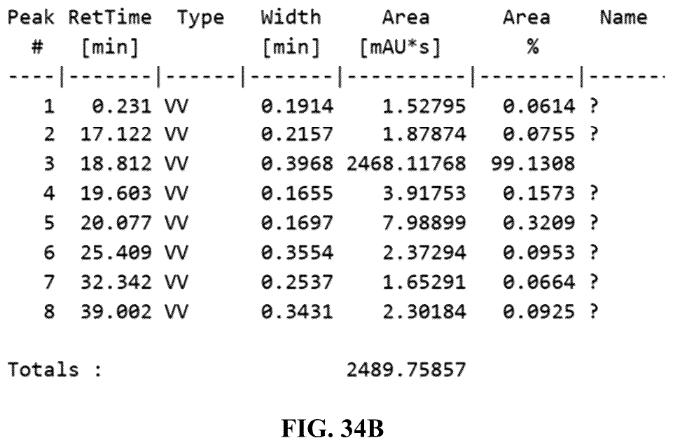

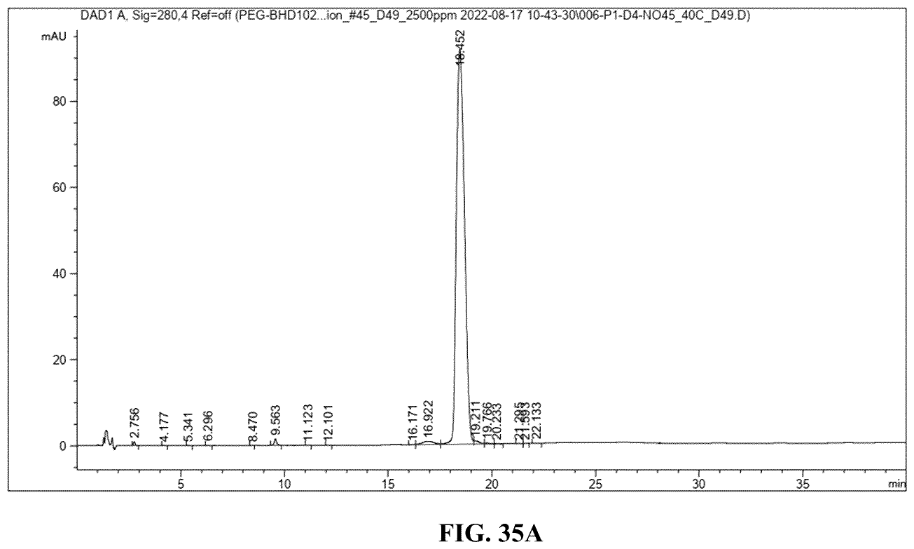

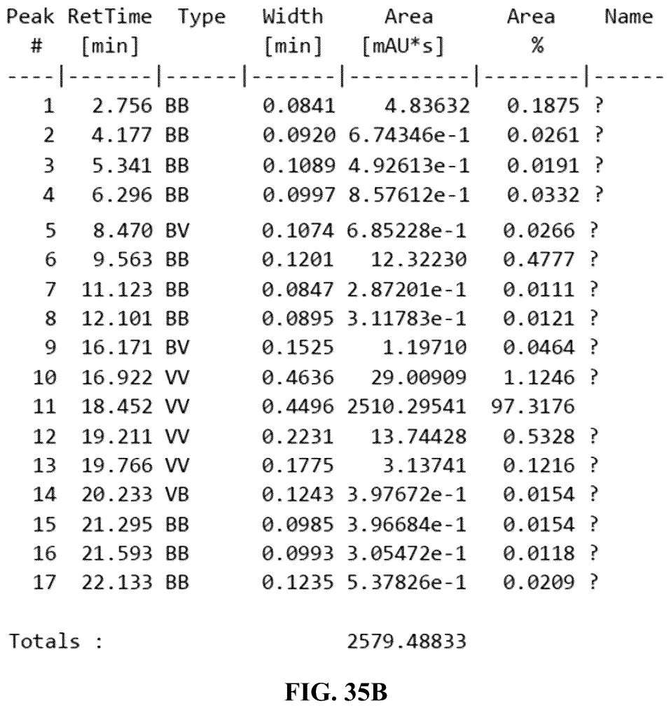

Formulations including salts such as KCl and NaCl were considerably decomposed even on Day 6 at 40° C. (see FIGS. 31A-31D). Formulations including PEG were also considerably decomposed even in a short period of time (see FIGS. 32A-32F). As a result of adding mannitol and trehalose, which are sucrose derivatives, as osmotic reagents, mannitol, trehalose, or a combination of mannitol and trehalose was stable, and thus mannitol and/or trehalose were set as the osmotic reagent (see FIGS. 33 and 34).

The osmotic pressure of the trehalose-only formulations according to the (PEG)-BHD1028 content (see Table 5) and the osmotic pressure of the trehalose-mannitol composite formulations according to the (PEG)-BHD1028 content (see Table 6) were measured, and reflecting the experimental results, the osmolarity including the 0.5% (PEG)-BHD1028 concentration was adjusted to about 300 mOsm/kg.

TABLE 5

| | | (PEG)- | (PEG)- | (PEG)- |
|---|---|---|---|---|
| | | BHD1028 | BHD1028 | BHD1028 |
| | Placebo | 0.5% | 1.5% | 4.5% |
| #1 | 296 mOsm/kg | 299 mOsm/kg | 310 mOsm/kg | 335 mOsm/kg |
| #2 | 293 mOsm/kg | 301 mOsm/kg | 309 mOsm/kg | 335 mOsm/kg |
| #3 | 295 mOsm/kg | 301 mOsm/kg | 307 mOsm/kg | 335 mOsm/kg |
| Mean | 294.67 | 300.33 | 308.67 | 335.00 |
| | mOsm/kg | mOsm/kg | mOsm/kg | mOsm/kg |

TABLE 6

| | | (PEG)- | (PEG)- | (PEG)- |
|---|---|---|---|---|
| | | BHD1028 | BHD1028 | BHD1028 |
| | Placebo | 0.5% | 1.5% | 4.5% |
| #1 | 289 mOsm/kg | 301 mOsm/kg | 309 mOsm/kg | 338 mOsm/kg |
| #2 | 290 mOsm/kg | 399 mOsm/kg | 312 mOsm/kg | 339 mOsm/kg |
| #3 | 291 mOsm/kg | 303 mOsm/kg | 307 mOsm/kg | 338 mOsm/kg |
| Mean | 290.00 | 301.00 | 309.33 | 338.33 |
| | mOsm/kg | mOsm/kg | mOsm/kg | mOsm/kg |

### 10-5. Shelf Life According to Stability Data

The shelf life was calculated based on the stability data reflecting the mean kinetic temperature (MKT). Theoretically, a substance at 40° C. is five times more active than at 25° C. Based on this calculation, the lifespan was calculated by multiplying the number of days immediately before the area % of a single impurity reached 1.0% by a factor of 5. The target shelf life was set at 6 months or longer at 25° C., and the stability of the trehalose-only formulation is expected to have a shelf life of about 7 months.

### Example 11: Phototoxicity Evaluation of (PEG)-BHD1028

To evaluate the phototoxicity of (PEG)-BHD1028, the potential phototoxicity of (PEG)-BHD1028 was measured in the BALB/3T3 clone A31 cells after UV exposure. The test substance and the positive control group (chlorpromazine hydrochloride; CPZ) were dissolved in a Hank's Balanced Salt Solution (HBSS) and then diluted with a Hank's Balanced Salt Solution (HBSS). The capacity range was set as shown in Table 7 below.

TABLE 7

| | UV | |
|---|---|---|
| | (+Irr)/(−Irr) | Dose (μg/ml) |
| (PEG)-BHD1028 | +Irr/−Irr | 0, 7.8, 15.6, 31.3, 62.5, 125, |
| | | 250, 500, 1000 |
| Chlorpromazine | +Irr | 0, 0.049, 0.098, 0.195, 0.39, |
| hydrochloride | | 0.78, 1.56, 3.13, 6.25 |
| | −Irr | 0, 0.78, 1.56, 3.13, 6.25, |
| | | 12.5, 25, 50, 100 |

The cells were trypsinized and counted. Thereafter, 1×10⁴ cells per well were seeded in a 96-well plate and cultured for approximately 24 hours before treating the test samples. One hour after the sample treatment, the plate was irradiated with a UV irradiation system (UVA, 365 nm). After UV irradiation, the plate was incubated for 24 hours. The cell viability of the cultured cells was confirmed by measuring the absorbance at 540 nm using the neutral red staining method.

As can be confirmed from the experimental results shown in Tables 8 and 9, as a result of confirming the cell viability according to the PIF (photo irradiation factor), MPE (mean photo effect), and the treatment of (PEG)-BHD1028, (PEG)-BHD1028 was classified as ‘non-phototoxic.’

TABLE 8

IC50 (μg/mL)

| Item | UV (−Irr) | UV (+Irr) | PIF | MPE |
|---|---|---|---|---|
| (PEG)- | >1000 | >1000 | 1.000 | 0.003 |
| BHD1028 | | | | |
| CPZ | 37.53 | 1.265 | 29.668 | 0.489 |

TABLE 9

| UV (−Irr) | | UV (+Irr) | |
|---|---|---|---|
| Dose (μg/mL) | Cell viability % | Dose (μg/mL) | Cell viability % |
| 0 | 100 | 0 | 100 |
| 7.8 | 104.70 | 7.8 | 108.02 |
| 15.6 | 107.47 | 15.6 | 110.97 |
| 31.3 | 110.81 | 31.3 | 112.03 |
| 62.5 | 112.51 | 62.5 | 111.09 |
| 125 | 112.57 | 125 | 111.64 |
| 250 | 112.63 | 250 | 110.01 |
| 500 | 112.68 | 500 | 111.84 |
| 1000 | 114.20 | 1000 | 109.28 |

In addition, chlorpromazine hydrochloride, which was a positive control group, showed positive results in PIF and MPE. Therefore, it was confirmed that (PEG)-BHD1028 causes no phototoxicity.

### Example 12: Confirmation of Dry Eye Syndrome Therapeutic Effect of (PEG)-BHD1028 Eye Drops

Through the eye drop test according to Example 9 above, an eye drop composition including (PEG)-BHD1028 0.5% (w/v), sodium EDTA 0.5% (w/v), hyaluronic acid salt 0.1% (w/v), and trehalose 9.0% (w/v) [0.5% (PEG)-BHD1028 eye drop composition] was prepared, and the dry eye syndrome therapeutic effect of the eye drop composition was confirmed in the experiment described below.

### 12-1. Evaluation of Corneal Damage Amelioration

Corneal Staining Score (CCS) is a clinical numerical evaluation method representing the ocular surface damage as a staining score, and it is an objective indicator of severity and grading. The experiment was performed by dropping 1 μL of 1% fluorescein solution into the eye of a subject (rabbit) with an experimental inflammatory eye disease, washing with 15 mL of physiological saline, and then observing under a slit-lamp biomicroscope to score and evaluate the grade of the epithelial damage (degree of fluorescent staining).

Corneal staining was graded using the NEI staining grade in five corneal regions (nasal, central, temporal, inferior, and superior) with a score from 0 to 3 (total 15 points) for each region (0=normal, 1=mild, 2=moderate, 3=severe). The statistical results were expressed as standard error of mean (±SEM), and the statistical significance of the differences between the experimental groups was determined using Student's t-test (GraphPad Prism 9.3.0). A p-value of 0.05 or less was considered as having statistical significance.

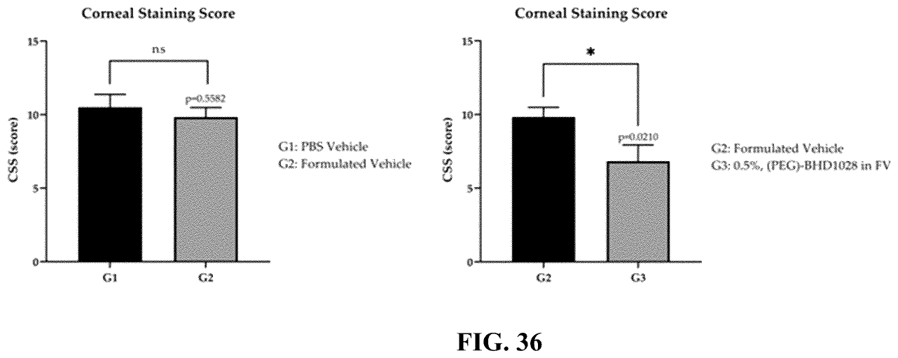

As a result of the experiment, as can be confirmed in FIG. 36, it was confirmed that there was no significant difference when the PBS vehicle was compared with the vehicle of the developed formulation. In addition, a statistically significant (*p<0.05) decrease of the numerical value was confirmed when the vehicle of developed formulation vehicle was compared with the 0.5% (PEG)-BHD1028 eye drop composition (0.5% (PEG)-BHD1028 in formulated vehicle), which is the developed eye drops.

In conclusion, in the case of the vehicle of the developed formulation, a clear placebo effect was confirmed, and it was confirmed that the 0.5% (PEG)-BHD1028 eye drop composition including the active medicinal ingredient had a statistically significant effect in ameliorating corneal damage. The reason for this ameliorating effect was confirmed through the anti-inflammatory response, cytoprotective effect, and increase in the number of goblet cells of the drug.

### 12-2. Corneal Thickness Evaluation in EDE Model

The eyes extracted by the histopathological evaluation method were washed with phosphate-buffered saline (PBS), fixed in the modified Davidson's solution, and embedded in paraffin (Histocore Arcadia, Leica, Wetzlar, Germany). Afterwards, the paraffin block was sliced into 6-μm thick sections, mounted on a microscope slide (HistoCore AUTOCUT, Leica, Germany), air-dried, and stained with hematoxylin-eosin (H&E). The stained tissue slides were imaged to obtain tissue staining data using an optical microscope/digital slide scanner (Axio Scan.Z1, Zeiss, Oberkochen, Germany), and the image measurement values were made by measuring the corneal epithelial thickness (λ40 magnification) using the Case Viewer (3DHISTECH, Hungary) software. All the values were determined as the average of the measurements obtained in three areas. The statistical results were expressed as standard error of mean (±SEM), and statistical significance of the differences between the experimental groups was determined using Student's t-test (GraphPad Prism 9.3.0). A p-value less than 0.05 was considered to be statistically significant.

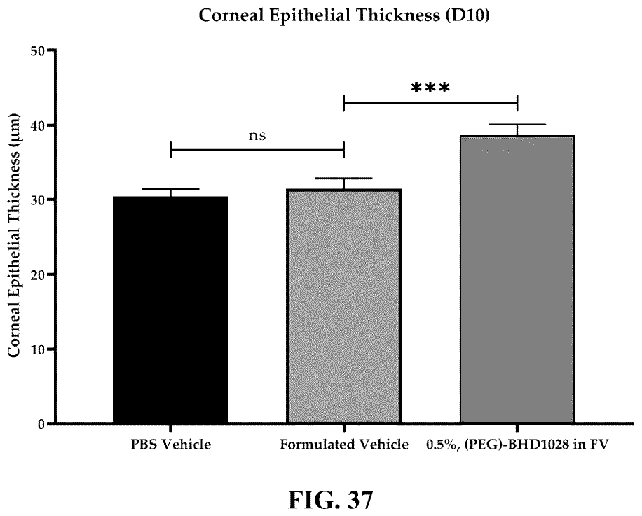

As a result of the experiment, as can be confirmed in FIG. 37, it was confirmed that there was no significant difference when the PBS vehicle was compared with the vehicle of the developed formulation, and in the case of the vehicle of the developed formulation, a clear placebo effect could be confirmed. When the vehicle of the developed formulation was compared with the 0.5% (PEG)-BHD1028 eye drop composition, which is the developed eye drops, a statistically significant (*** p<0.001) increase in the corneal thickness was found. In a chronically stressed corneal epithelium, tear evaporation may increase, which increases the osmolarity of tears, thereby thinning the cornea. The developed eye drop including the active medicinal ingredient showed a tissue cell protection effect through statistically significant regeneration of the corneal epithelium, and amelioration of the corneal damage could be confirmed.

### 12-3. Confirmation of Anti-Inflammatory Effect

The ocular and lacrimal gland tissues extracted by the histopathological evaluation method were washed with phosphate-buffered saline (PBS), fixed in the modified Davidson's solution, and then embedded in paraffin (Histocore Arcadia, Leica, Wetzlar, Germany). Afterwards, the paraffin block was sliced into 6-μm thick sections, mounted on a microscope slide (HistoCore AUTOCUT, Leica, Germany), air-dried, and stained with hematoxylin-eosin (H&E). The stained tissue slides were imaged to obtain tissue staining data using an optical microscope/digital slide scanner (Axio Scan.Z1, Zeiss, Oberkochen, Germany), and in order to investigate the degree of corneal inflammatory cell infiltration, the stromal layer, conjunctiva, and lacrimal gland areas were selected (400 μm×300 μm) on the Case Viewer (3DHISTECH, Hungary) software program, and the number of cell nuclei was measured using the Image J (NIH, Bethesda, MA, USA) software program. All the values were determined as the average value of the measurements obtained from three areas per slide. The statistical results were expressed as standard error of mean (±SEM), and the statistical significance of the differences between the experimental groups was determined using Student's t-test (GraphPad Prism 9.3.0). A p-value less than 0.05 was considered to be statistically significant.

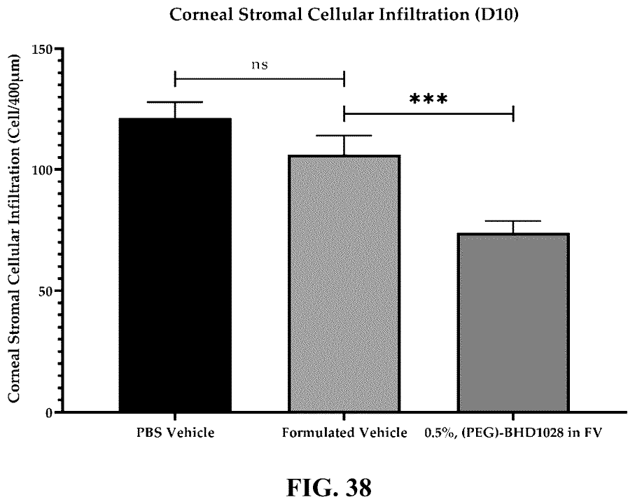

As a result of the experiment, as can be confirmed in FIG. 38, whether the test substance could reduce the inflammatory response of the cornea caused by dry eye syndrome was evaluated, and the results were obtained by measuring the degree of inflammatory cell infiltration in the corneal stromal layer. It was confirmed that there was no significant difference when the PBS vehicle was compared with the vehicle of the developed formulation. In addition, when the vehicle of the developed formulation was compared with the 0.5% (PEG)-BHD1028 eye drop composition, which is the developed eye drops, a statistically significant (*** p<0.001) decrease was found in inflammatory cell infiltration.

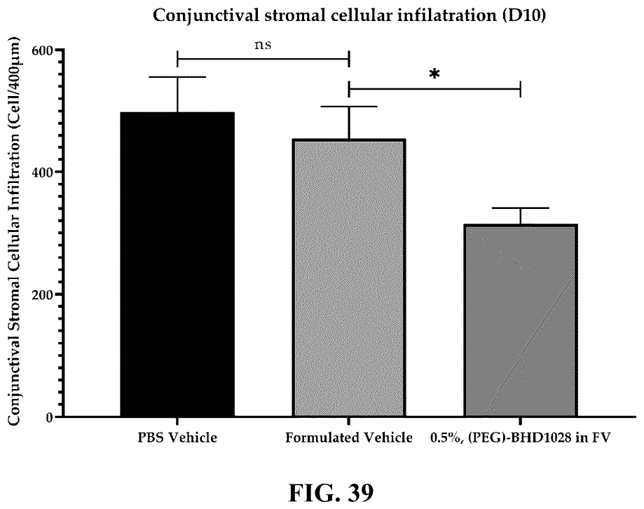

In addition, as shown in FIG. 39, whether the test substance could reduce the inflammatory response of the conjunctiva caused by dry eye syndrome was evaluated to measure the degree of inflammatory cell infiltration in the conjunctiva. It was confirmed that there was no significant difference when the PBS vehicle was compared with the vehicle of the developed formulation. In addition, when the vehicle of the developed formulation was compared with the 0.5% (PEG)-BHD1028 eye drop composition, which is the developed eye drops, a statistically significant (*p<0.05) decrease was found in inflammatory cell infiltration.

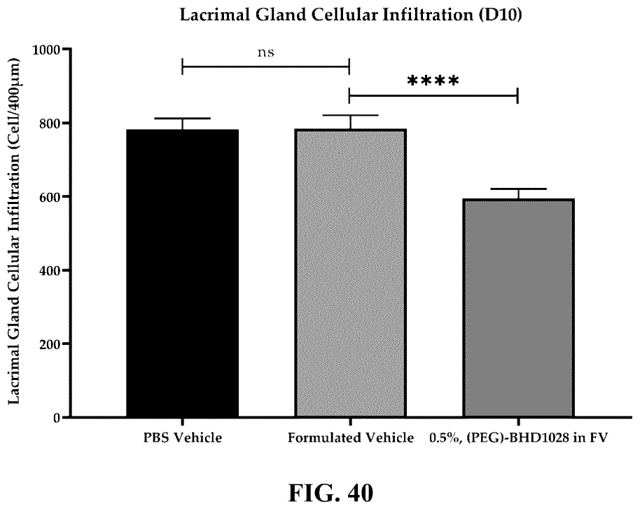

In addition, as shown in FIG. 40, whether the test substance could reduce the inflammatory response of the lacrimal gland caused by dry eye syndrome was evaluated to measure the degree of inflammatory cell infiltration in the lacrimal gland. It was confirmed that there was no significant difference when the PBS vehicle was compared with the vehicle of the developed formulation. In addition, when the vehicle of the developed formulation was compared with the 0.5% (PEG)-BHD1028 eye drop composition, which is the developed eye drops, a statistically significant (**** p<0.0001) decrease was found in inflammatory cell infiltration.

When the tear production decreases, the tear clearance rate decreases, and various inflammatory mediators appear on the ocular surface. When evaluated from the perspective of an inflammatory disease in which an inflammatory response thereby begins and dry eyes occur, in the case of the vehicle of the developed formulation, a clear placebo effect was confirmed in the cornea, conjunctiva, and lacrimal gland tissue. In addition, it was confirmed that the 0.5% (PEG)-BHD1028 eye drop composition including the active medicinal ingredient ameliorated the eye damage through a statistically significant anti-inflammatory response.

### 12-4. Confirmation of Antiapoptotic Effect

The eyeballs extracted by the histopathological evaluation method were washed with phosphate-buffered saline (PBS), fixed in the modified Davidson's solution, and then embedded in paraffin (Histocore Arcadia, Leica, Wetzlar, Germany). Afterwards, the paraffin block was sliced into 6-μm thick sections, mounted on a microscope slide (HistoCore AUTOCUT, Leica, Germany), air-dried, and stained with TUNEL. The stained tissue slides were imaged to obtain tissue staining data using an optical microscope/digital slide scanner (Axio Scan.Z1, Zeiss, Oberkochen, Germany), and in order to investigate the degree of corneal cell death, the corneal and conjunctival areas were selected (400 μm×300 μm) on the Case Viewer (3DHISTECH, Hungary) software program, and the TUNEL positive/negative area ratio was measured using the Image J (NIH, Bethesda, MA, USA) software program. All the values were determined as the average value of the measurements obtained from three areas per slide. The statistical results were expressed as standard error of mean (±SEM), and the statistical significance of the differences between the experimental groups was determined using Student's t-test (GraphPad Prism 9.3.0). A p-value less than 0.05 was considered to be statistically significant.

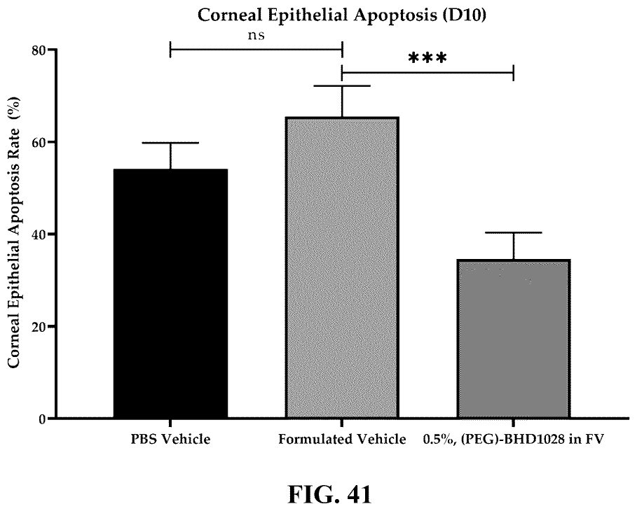

As shown in FIG. 41, to evaluate whether the test substance could reduce the cell death of the corneal epithelium caused by dry eye syndrome, the ratio occupied by the TUNEL positive cells in all the corneal epithelial cells was measured. It was confirmed that there was no significant difference when the PBS vehicle was compared with the vehicle of the developed formulation. In addition, when the vehicle of the developed formulation was compared with the 0.5% (PEG)-BHD1028 eye drop composition, which is the developed eye drops, a statistically significant (**** p<0.0001) decrease was found in cell death.

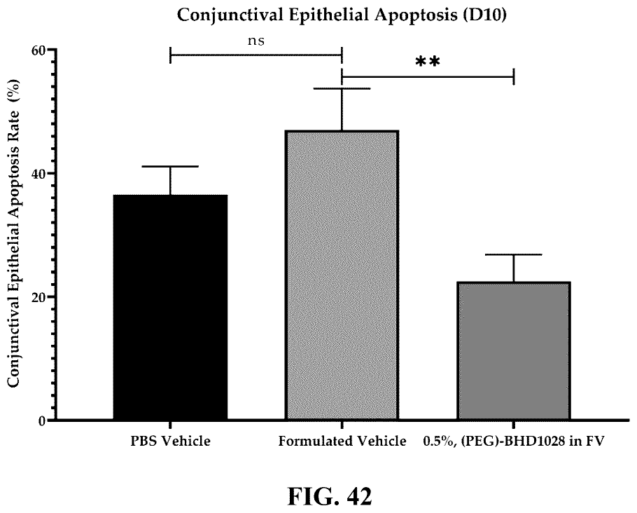

In addition, as shown in FIG. 42, to evaluate whether the test substance could reduce the cell death of the conjunctival epithelium caused by dry eye syndrome, the ratio occupied by the TUNEL positive cells in all the conjunctival epithelial cells was measured. It was confirmed that there was no significant difference when the PBS vehicle was compared with the vehicle of the developed formulation. In addition, when the vehicle of the developed formulation was compared with the 0.5% (PEG)-BHD1028 eye drop composition, which is the developed eye drops, a statistically significant (** p<0.01) decrease was found in cell death.

In both the cornea and conjunctiva, in the case of the vehicle of the developed formulation, a clear placebo effect was confirmed. In the developed eye drops including the active drug ingredient (0.5% (PEG)-BHD1028 eye drop composition), the fundamental cell damage therapeutic effect was confirmed through the statistically significant inhibition of cell death.

### 12-5. Measurement of Goblet Cell Counts in Conjunctiva

The eyeballs extracted by the histopathological evaluation method were washed with phosphate-buffered saline (PBS), fixed in the modified Davidson's solution, and then embedded in paraffin (Histocore Arcadia, Leica, Wetzlar, Germany). Afterwards, the paraffin block was sliced into 6-μm thick sections, mounted on a microscope slide (HistoCore AUTOCUT, Leica, Germany), air-dried, and stained with periodic acid-Schiff (PAS). The stained tissue slides were imaged to obtain tissue staining data using an optical microscope/digital slide scanner (Axio Scan.Z1, Zeiss, Oberkochen, Germany), and in order to investigate the degree of cell death, the conjunctival area was selected (400 μm×300 μm) on the Case Viewer (3DHISTECH, Hungary) software program, and the number of conjunctival goblet cells was measured using the Image J (NIH, Bethesda, MA, USA) software program. All the values were determined as the average value of the measurements obtained from three areas per slide. The statistical results were expressed as standard error of mean (±SEM), and the statistical significance of the differences between the experimental groups was determined using Student's t-test (GraphPad Prism 9.3.0). A p-value less than 0.05 was considered to be statistically significant.

The tear film on the surface of the eyes consists of three major components, which are a lipid layer, an aqueous layer, and a mucus layer, starting from the outer layer, and it serves as a protective film for the eyes. When even one of these components is lacking, the tear film becomes unstable, and tears evaporate easily. The mucus layer is a tear layer made of mucus that covers the surface of the cornea, and it is produced from the goblet cells of the conjunctiva and plays the role of preventing the evaporation of the aqueous layer.

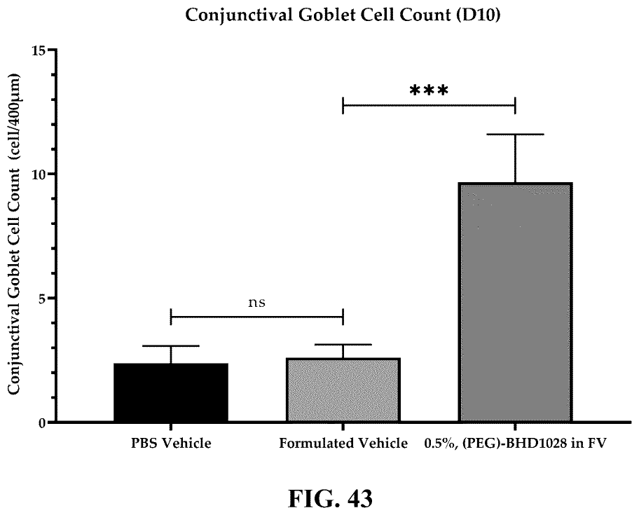

As shown in FIG. 43, to evaluate whether the test substance has a cell proliferation effect in the dysfunction and cell death of the conjunctival goblet cells caused by dry eye syndrome, after staining the ocular tissues with periodic acid-Schiff (PAS), the number of goblet cells in the conjunctival epithelium layer was measured. It was confirmed that there was no significant difference when the PBS vehicle was compared with the vehicle of the developed formulation, and when the vehicle of the developed formulation was compared with the 0.5% (PEG)-BHD1028 eye drop composition, a statistically significant (*** p<0.001) increase was found in the number of the goblet cells. In the case of the vehicle of the developed formulation, a clear placebo effect was confirmed. In the developed eye drops including the active drug ingredient (0.5% (PEG)-BHD1028 eye drop composition), a statistically significant increase of the goblet cells was confirmed. A majority of the patients exhibit subjective symptoms in the stage of ocular surface cell loss, and this loss of goblet cells is one of the key causes of the vicious cycle of dry eyes. Therefore, it was confirmed that the developed eye drop is a therapeutic method for numerical restoration of the lost goblet cells.

What is claimed is:

1. A method for treating ocular diseases comprising step of:
administering to a subject, a pharmaceutical composition comprising a peptide including SEQ ID NO: 1 (Xaa1-Tyr-Phe-Ala-Tyr-His-Pro-Asn-Ile-Pro-Gly-Leu-Xaa2-Tyr-Phe) as an active ingredient,
wherein in the sequence of SEQ ID NO: 1, the Xaa1 and Xaa2 is any one selected from the group consisting of tyrosine, tryptophan, phenylalanine, and non-natural amino acids having the same properties as these.

2. The method of claim 1, wherein a linking compound of X-L1 is further linked to an N terminus of an amino acid sequence of the SEQ ID NO: 1,
wherein the X is any one selected from the group consisting of 1 to 10 amino acids;
polyethylene glycol in a linear or branched form having a weight of 1 to 200 kDa; a lipophilic compound; and a peptide transduction domain, and
wherein the L1 is a compound that plays the role of a single bond or a link connecting an N terminus of the SEQ ID NO: 1 and the X above.

3. The method of claim 2, wherein the X is polyethylene glycol in a linear or branched form having a weight of 1 to 200 kDa.

4. The method of claim 1, wherein the ocular disease is one or more diseases selected from the group consisting of keratoconjunctivitis sicca (KCS), atopic keratoconjunctivitis (AKC), vernal keratoconjunctivitis (VKC), glaucoma, keratitis, corneal epithelium erosion, uveitis, intraocular inflammation, dry eye syndrome, dry-eye syndrome ocular infections, ocular infections, ocular allergy, corneal or conjunctival lesions, diabetic macular edema, and age-related macular degeneration.

5. The method of claim 1, further comprising an L2-Z linking compound at a C terminus of the amino acid sequence of the SEQ ID NO: 1,
wherein the Z is any one selected from the group consisting of 1 to 10 amino acids; polyethylene glycol in a linear or branched form having a weight of 1 to 200 kDa; a lipophilic compound; and a peptide transduction domain, and
wherein the L2 is a compound that plays the role of a single bond or a link connecting a C terminus of the SEQ ID NO: 1 and the Z.

6. The method of claim 1, wherein the SEQ ID NO: 1 is any one selected from the group consisting of Tyr-Tyr-Phe-Ala-Tyr-His-Pro-Asn-Ile-Pro-Gly-Leu-Tyr-Tyr-Phe (SEQ ID NO: 2); Trp-Tyr-Phe-Ala-Tyr-His-Pro-Asn-Ile-Pro-Gly-Leu-Trp-Tyr-Phe (SEQ ID NO: 3); and Phe-Tyr-Phe-Ala-Tyr-His-Pro-Asn-Ile-Pro-Gly-Leu-Phe-Tyr-Phe (SEQ ID NO: 4).

7. The method of claim 1, wherein the pharmaceutical composition is in a form of eye drops, artificial tears, gels or ointments.

8. The method of claim 1, wherein the pharmaceutical composition includes ethylene-diamine-tetraacetic acid (EDTA) or EDTA sodium salt.

9. The method of claim 1, wherein the pharmaceutical composition includes hyaluronic acid or hyaluronic acid salt.

10. The method of claim 1, wherein the pharmaceutical composition has a viscosity of 6 to 14 cP.

11. The method of claim 1, wherein the pharmaceutical composition has pH of 6.0 to 8.0.

12. The method of claim 1, wherein the pharmaceutical composition includes trehalose or mannitol.

13. The method of claim 1, wherein the pharmaceutical composition has an osmolality of 280 to 340 mOsm/kg.

14. The method of claim 1, wherein the pharmaceutical composition includes 0.1% to 0.9% (w/v) of ethylene-diamine-tetraacetic acid (EDTA), 0.05% to 0.2% (w/v) of sodium hyaluronate, and 4.0% to 16.0% (w/v) of trehalose.
